(19) United States
(12) Patent Application Publication
(10) Pub. No.: US 2004/0038856 A1
(43) Pub. Date: Feb. 26, 2004

(54) Treatment of fibroproliferative disorders using TGF-beta inhibitors

(76) Inventors: Sarvajit Chakravarty, Sunnyvale, CA (US); Sundeep Dugar, San Jose, CA (US); Linda S. Higgins, Palo Alto, CA (US); Ann M. Kapoun, Palo Alto, CA (US); David Y. Liu, Palo Alto, CA (US); Andrew A. Protter, Palo Alto, CA (US); George F. Schreiner, Los Altos, CA (US); Thomas-Toan Tran, Sunnyvale, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/440,428

(22) Filed: May 16, 2003

Related U.S. Application Data
(60) Provisional application No. 60/381,720, filed on May 17, 2002.

(51) Int. Cl. A61K 31/00

(52) U.S. Cl. 514/1

(57) ABSTRACT

The invention concerns methods of treating fibroproliferative disorders associated with TGF-β signaling, by administering non-peptide small molecule inhibitors of TGF-β specifically binding to the type I TGF-β receptor (TGFβ-R1). Preferably, the inhibitors are quinazoline derivatives. The invention also concerns methods for reversing the effect of TGF-β-mediated cell activation on the expression of a gene associated with fibrosis, comprising contacting a cell or tissue in which the expression of such gene is altered as a result of TGF-β-mediated cell activation, with a non-peptide small molecule inhibitor of TGF-β, specifically binding a TGFβ-R1 receptor kinase present in the cell or tissue.

### CROSS-REFERENCE TO RELATED APPLICATIONS

This is a non-provisional application filed under 37 CFR 1.53(b), claiming priority under USC Section 119(e) to provisional Application Ser. No. 60/381720 filed May 17, 2002.

### BACKGROUND OF THE INVENTION

1. Field of the Invention

The present invention concerns methods of treatment using transforming growth factor β (TGF-β) inhibitors. More specifically, the invention concerns methods of treating fibroproliferative disorders associated with TGF-β signaling, by administering TGF-β inhibitors specifically binding to the type I TGF-β receptor (TGFβ-R1).

2. Description of the Related Art

Transforming growth factor-beta (TGF-β) denotes a family of proteins, TGF-β1, TGF-β2, and TGF-β3, which are pleiotropic modulators of cell growth and differentiation, embryonic and bone development, extracellular matrix formation, hematopoiesis, immune and inflammatory responses (Roberts and Sporn Handbook of Experimental Pharmacology (1990) 95:419-58; Massague et al. Ann Rev Cell Biol (1990) 6:597-646). Other members of this superfamily include activin, inhibin, bone morphogenic protein, and Mullerian inhibiting substance. TGF-β initiates intracellular signaling pathways leading ultimately to the expression of genes that regulate the cell cycle, control proliferative responses, or relate to extracellular matrix proteins that mediate outside-in cell signaling, cell adhesion, migration and intercellular communication..

TGF-β exerts its biological activities through a receptor system including the type I and type II single transmembrane TGF-β receptors (also referred to as receptor subunits) with intracellular serine-threonine kinase domains, that signal through the Smad family of transcriptional regulators. Binding of TGF-β to the extracellular domain of the type II receptor induces phosphorylation and activation of the type I receptor (TGFβ-R1) by the type II receptor (TGFβ-R2). The activated TGFβ-R1 phosphorylates a receptor-associated co-transcription factor Smad2/Smad3, thereby releasing it into the cytoplasm, where it binds to Smad4. The Smad complex translocates into the nucleus, associates with a DNA-binding cofactor, such as Fast-1, binds to enhancer regions of specific genes, and activates transcription. The expression of these genes leads to the synthesis of cell cycle regulators that control proliferative responses or extracellular matrix proteins that mediate outside-in cell signaling, cell adhesion, migration, and intracellular communication. Other signaling pathways like the MAP kinase-ERK cascade are also activated by TGF-β signaling. For review, see, e.g. Whitman, Genes Dev. 12:2445-62 (1998); and Miyazono et al., Adv. Immunol. 75:111-57 (2000), which are expressly incorporated herein by reference.

### SUMMARY OF THE INVENTION

The invention concerns the treatment of fibroproliferative diseases. In particular, the invention concerns the treatment of fibroproliferative diseases with small molecule inhibitors specifically binding a type 1 TGF-β receptor (TGFβ-R1).

In one aspect, the invention concerns a method for the treatment of a fibroproliferative disease, comprising

(a) identifying a non-peptide small molecule, selectively binding to a TGFβ-R1 kinase receptor; and

(b) administering an effective amount of such molecule to a mammalian subject diagnosed with the fibroproliferative disease.

In another aspect, the invention concerns a method for reversing the effect of TGF-β-mediated cell activation on the expression of a gene associated with fibrosis, comprising contacting a cell or tissue in which the expression of such gene is altered as a result of TGF-β-mediated cell activation, with a non-peptide small molecule inhibitor of TGF-β, specifically binding a TGFβ-R1 receptor kinase present in the cell or tissue. The targeted cell or tissue can be in vivo or as part of an in vitro culture. Preferably, the gene is associated with fibrosis, and can be overexpressed or underexpressed as a result of TGF-β -mediated cell activation.

Genes overexpressed as a result of TGF-β-mediated cell activation include, for example, fibronectin, collagen, type I, alpha 2 (COL1A2); collagen, type V, alpha 2 (COL5A2); connective tissue growth factor (CTGF); thrombospondin 1 (THBS1); hexabrachion (HXB); tissue inhibitor of metalloproteinase 1 (TIMP-1); tissue inhibitor of metalloproteinase 3 (TIMP3); plasminogen activator inhibitor-1 (PAI-1); an collagen, type III, alpha-1 (COL3A1).

Genes underexpressed as a result of TGF-β-mediated cell activation include, for example, platelet-derived growth factor receptor-α (PDGFRα).

In another embodiment, the invention concerns a method for determining the possibility of a positive response of a subject diagnosed with a fibroproliferative disease associated with TGF-β-mediated cell activation to treatment with a TGF-β inhibitor specifically binding the TGFβ-R1 receptor, comprising

(a) determining in a biological sample obtained from said subject the expression level of one or more genes selected from the group consisting of fibronectin, collagen, type I, alpha 2 (COL1A2); collagen, type V, alpha 2 (COL5A2); connective tissue growth factor (CTGF); thrombospondin 1 (THBS1); hexabrachion (HXB); tissue inhibitor of metalloproteinase 1 (TIMP-1); tissue inhibitor of metalloproteinase 3 (TIMP3); plasminogen activator inhibitor-1 (PAI-1); collagen, type III, alpha-1 (COL3A1); and platelet-derived growth factor receptor-α (PDGFRα), compared with expression in a sample obtained from a normal subject; and

(b) indicating a positive response, if one or more of such genes are differentially expressed.

In a different aspect, the invention concerns a method of diagnosing a patient with a fibroproliferative disease, comprising

(a) determining in a biological sample obtained from said patient the expression level of one or more genes selected from the group consisting of fibronectin, collagen, type I, alpha 2 COL1A2); collagen, type V, alpha 2 (COL5A2); connective tissue growth factor (CTGF); thrombospondin 1 (THBS1); hexabrachion (HXB); tissue inhibitor of metalloproteinase 1 (TIMP-1); tissue inhibitor of metalloproteinase 3 (TIMP3); plasminogen activator inhibitor-1 (PAT-1); platelet-derived growth factor receptor-α (PDGFRα); and collagen, type III, alpha 1 (COL3A1), compared with expression in a normal sample; and

(b) diagnosing said patient with a fibroproliferative disease if one or more of said genes are differentially expressed.

In a further embodiment, the invention concerns the treatment of a patient diagnosed with a fibroproliferative disease, comprising administering to said patient an effective amount of a small molecule selectively binding to a TGFβ-R1 kinase receptor and capable of reversing the effect of TGF-β-mediated cell activation on the expression of a gene associated with fibrosis.

In all embodiments, the fibroproliferative disease includes renal, hepatic, pulmonary, cardiovascular, eye, opthalmolized, and dermatological disorders associated with enhanced TGF-β receptor activation and excessive fibrosis or sclerosis.

Exemplary fibroproliferative diseases include, without limitation, glomerulonephritis (GN); diabetic nephropathy; renal interstitial fibrosis; renal fibrosis resulting from complications of drug exposure; HIV-associated nephropathy; transplant necropathy; liver cirrhosis due to all etiologies; disorders of the biliary tree; hepatic dysfunction attributable to infections; pulmonary fibrosis; adult respiratory distress syndrome (ARDS); chronic obstructive pulmonary disease (COPD); idiopathic pulmonary fibrosis (IPF); acute lung injury (ALI); pulmonary fibrosis due to infectious or toxic agents; congestive heart failure; dilated cardiomyopathy; myocarditis; vascular stenosis; progressive systemic sclerosis; polymyositis; scleroderma; dermatomyositis; fascists; Raynaud's syndrome, rheumatoid arthritis; proliferative vitreoretinopathy; fibrosis associated with ocular surgery; and excessive or hypertrophic scar and/or keloid formation in the dermis occurring during wound healing resulting from trauma or surgical wounds.

The subject treated can be any mammal but preferably is human.

### BRIEF DESCRIPTION OF THE DRAWINGS

FIG. 1 illustrates the inhibition of TGF-β induced Smad2/3 translocation to the nucleus of rat lung fibroblasts (RLF).

FIG. 2 shows the effect of an inhibitor of the invention on PAI-1 secretion from human lung fibroblasts (HLF) stimulated with TGF-β at 48 hours.

FIG. 3 shows the effect of an inhibitor of the invention on CTGF intracellular protein expression from RLF in the time course of 48 hours.

FIG. 4 shows the inhibition of TGF-β-induced PAI-1 protein expression by an inhibitor of the invention in Hep G2 cells.

FIG. 5 is a table showing TGF-β stimulated fibrotic genes affected by inhibitors of the invention.

FIG. 6 shows that TGF-β-induced gene expression of osteopontin is reversed by an inhibitor of the invention in Rat Whole Blood Cells at 4 hours.

FIG. 7 shows the plasma concentrations of an inhibitor of the invention in a bleomycin rat model of pulmonary fibrosis.

FIG. 8 shows the percent body weight change of animals in a bleomycin rat model of pulmonary fibrosis from day 0.

FIG. 9 shows the total interleukin-6 (IL-6) concentration in the bronchoalveolar lavage fluids (BALF).

FIG. 10 shows plasminogen activator inhibitor-1 (PAI-1) mRNA expression in the lung tissues of rats.

FIG. 11 shows connective tissue growth factor (CTGF) mRNA expression in the lung tissues of rats.

FIG. 12 shows tissue inhibitor of metalloproteinase 1 (TIMP-1) mRNA expression in the lung tissues of rats.

FIG. 13 shows fibronectin mRNA expression in the lung tissues of rats.

FIG. 14 shows inhibition of α-SMA protein expression by a test compound.

FIG. 15 shows inhibition of IL-6 protein expression by a test compound

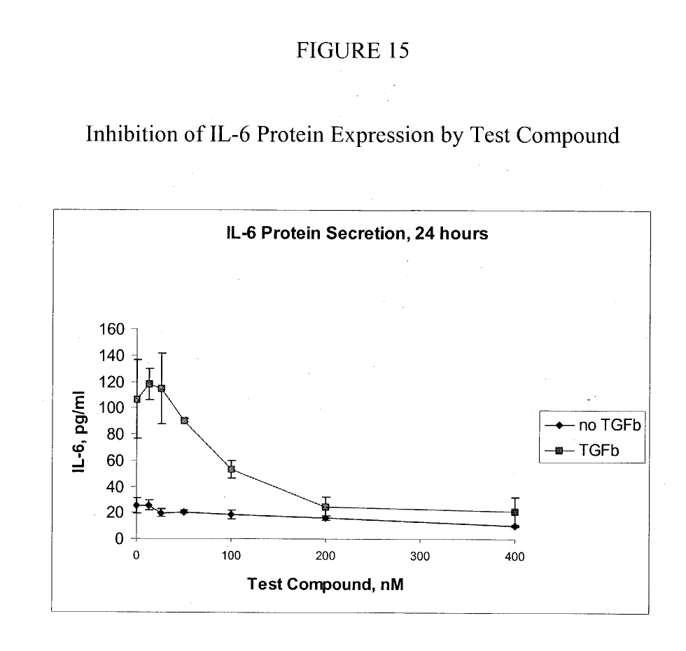

FIG. 16 shows inhibition of PAI-I protein expression by a test compound.

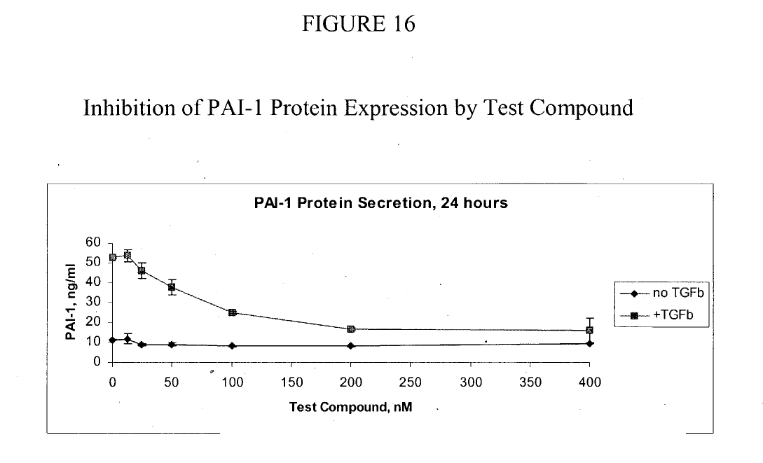

FIG. 17 shows inhibition of Pro-Col 1 C-peptide expression by a test compound.

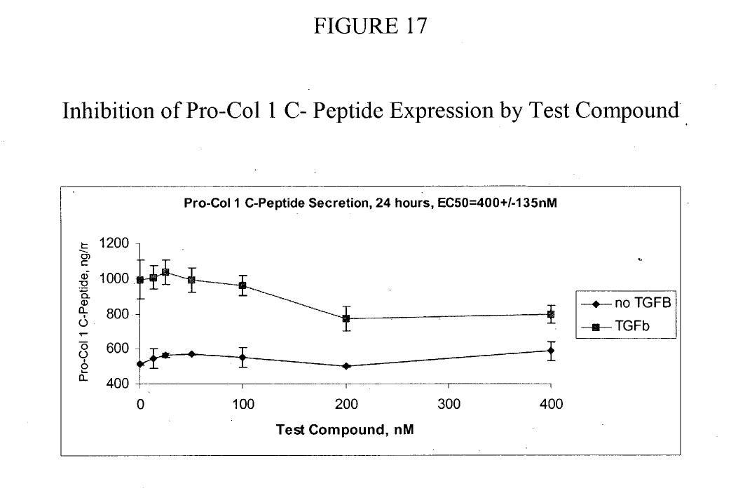

FIG. 18 shows a test compound blocking TGF-β induced Smad2 phosphorylation in HLF cells.

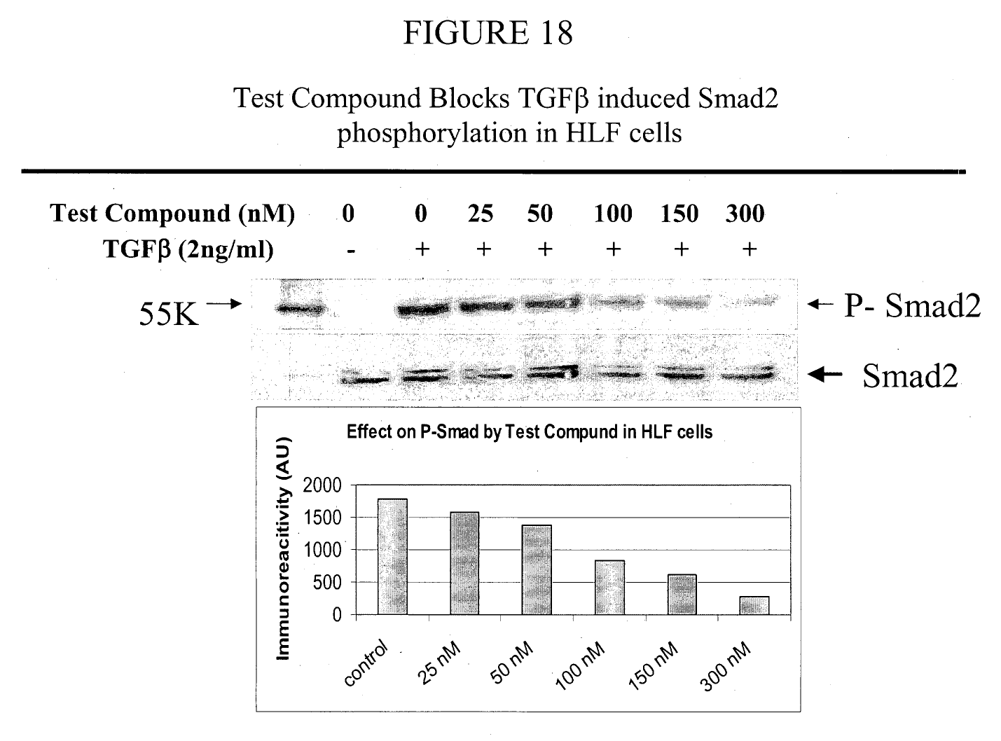

FIG. 19 shows a test compound inhibiting TGF-β induced Smad2 nuclear translocation in HLF cells.

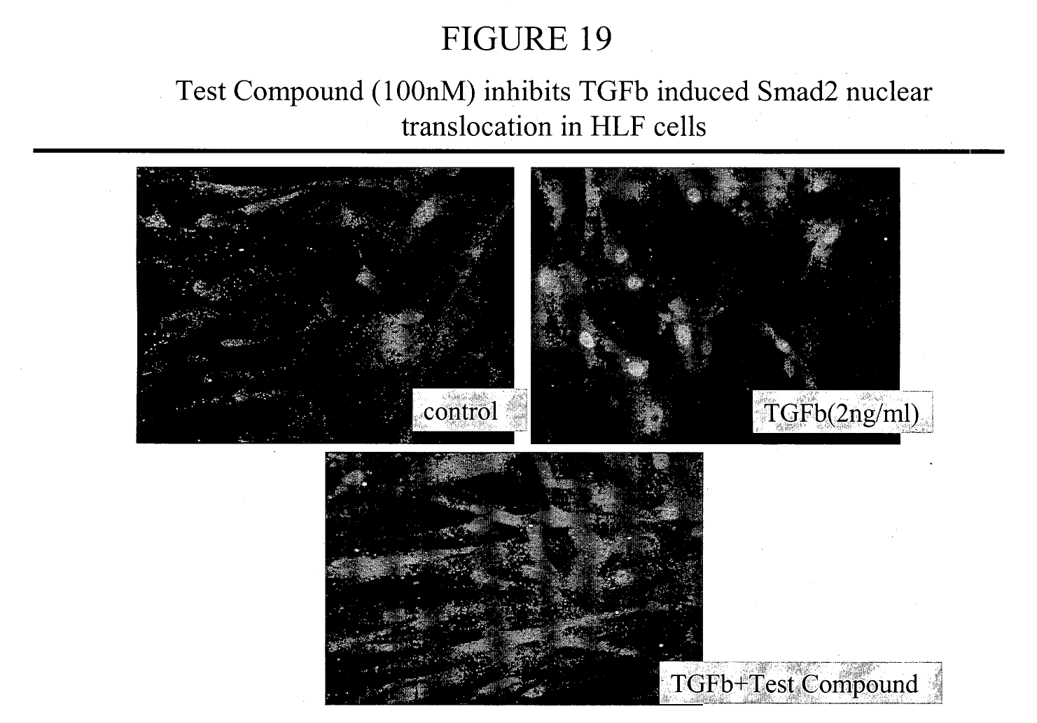

FIG. 20 shows a test compound inhibiting TGF-β induced Smad3 nuclear translocation in HLF cells.

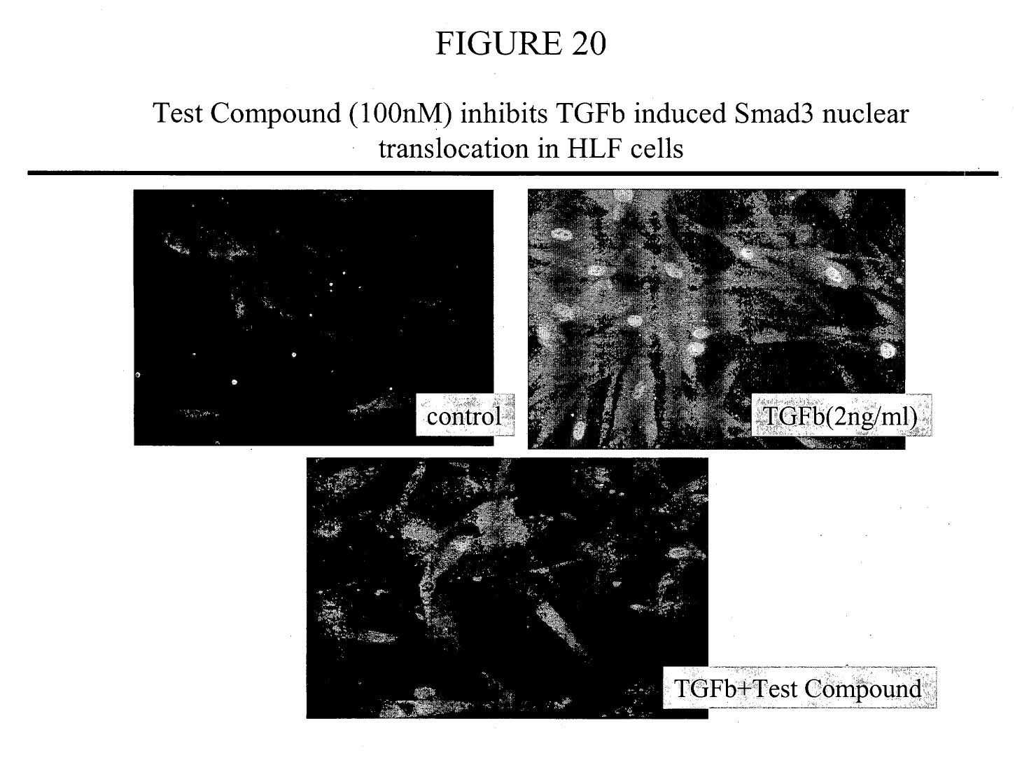

FIG. 21 shows inhibition of various kinases by a test compound.

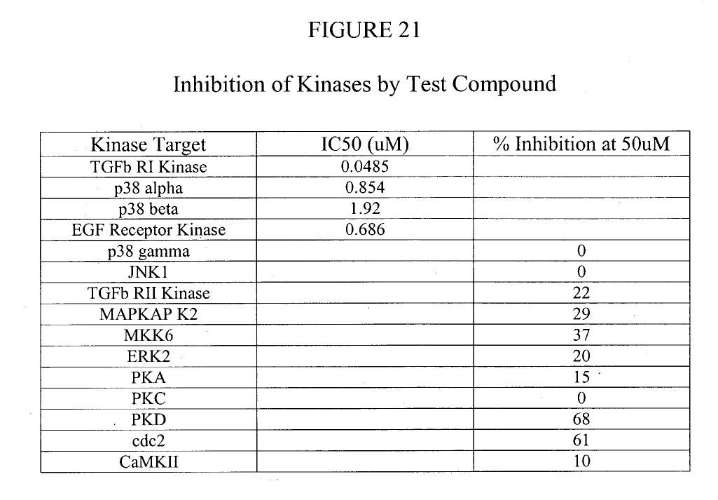

FIG. 22 shows inhibition of activin-induced hemaglobin production in K562 cells by test compounds.

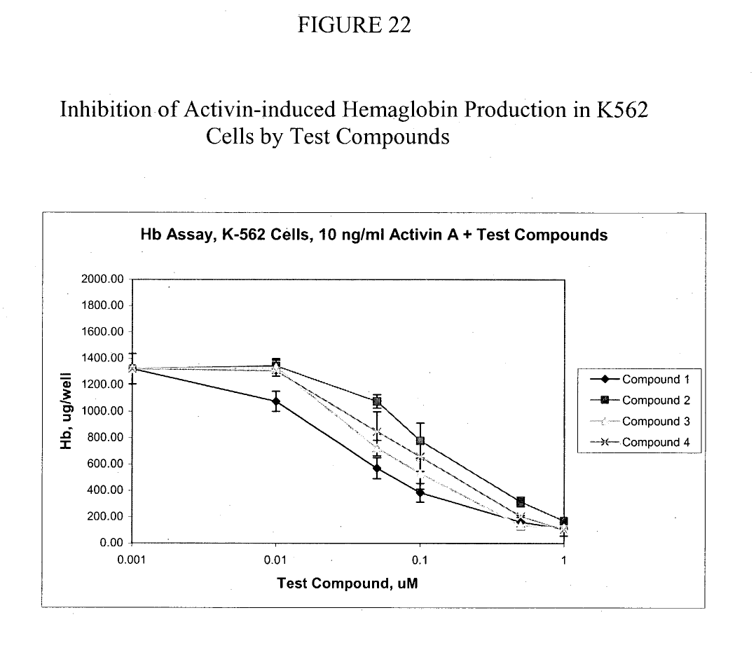

FIG. 23 shows CTGF expression in HLF cells when treated with test compounds.

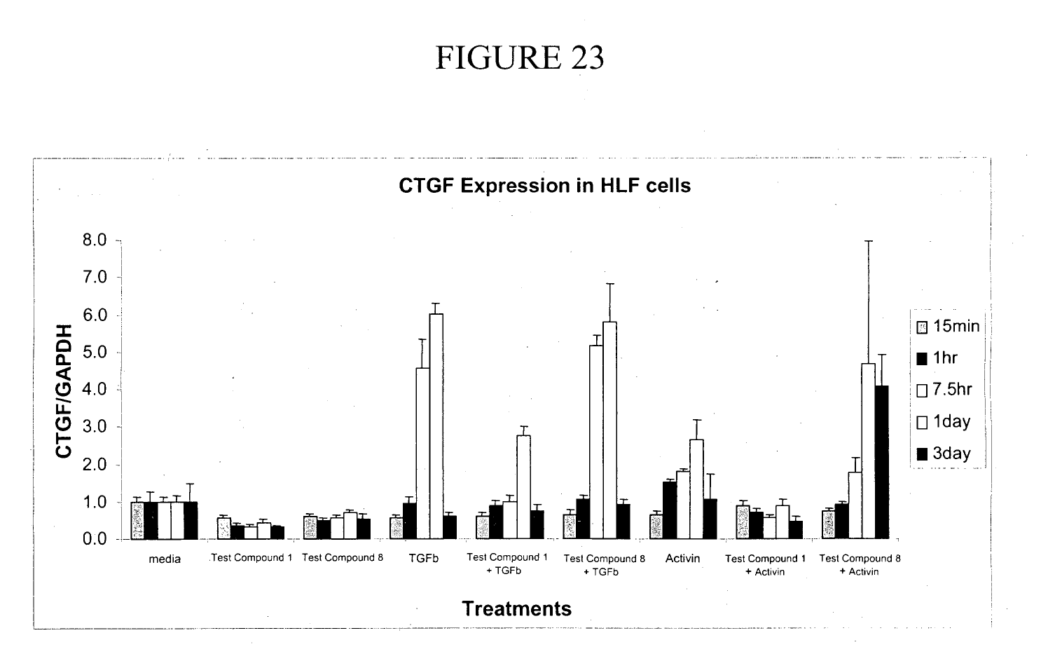

FIG. 24 shows PAI-1 expression in HLF cells when treated with test compounds.

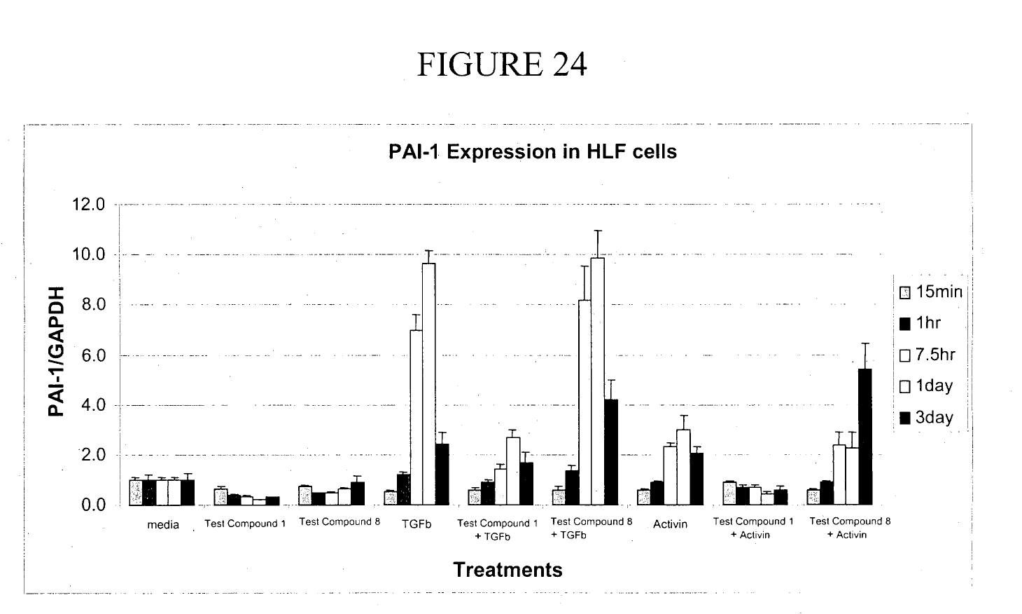

FIG. 25 shows CTGF mRNA expression in HLF cells when treated with test compounds.

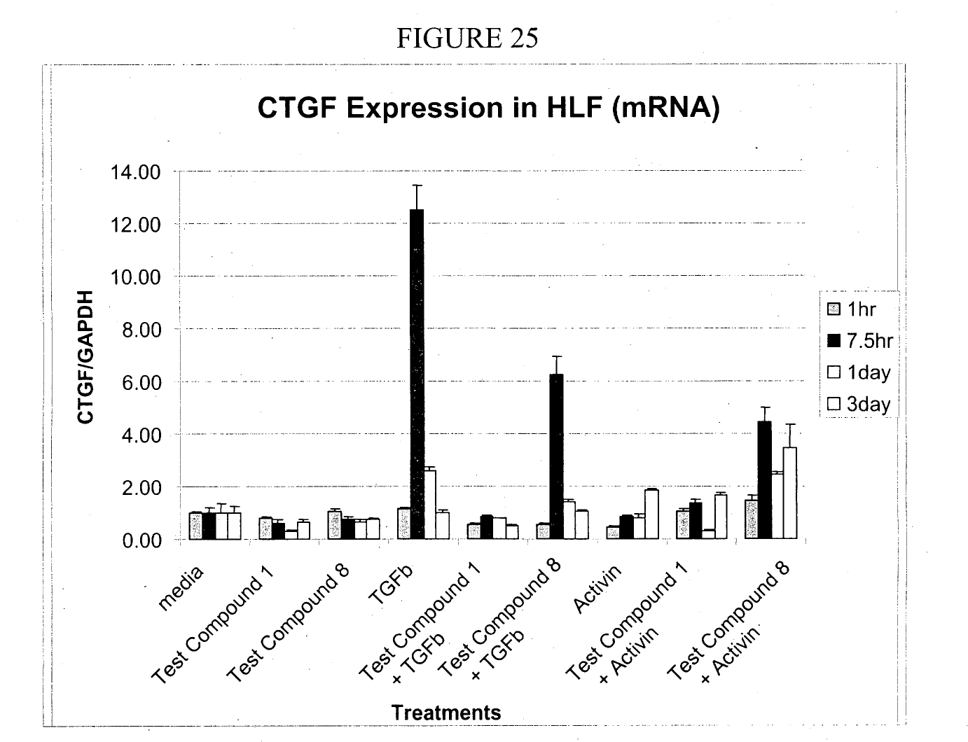

FIG. 26 shows PAI-1 mRNA expression in HLF cells when treated with test compounds.

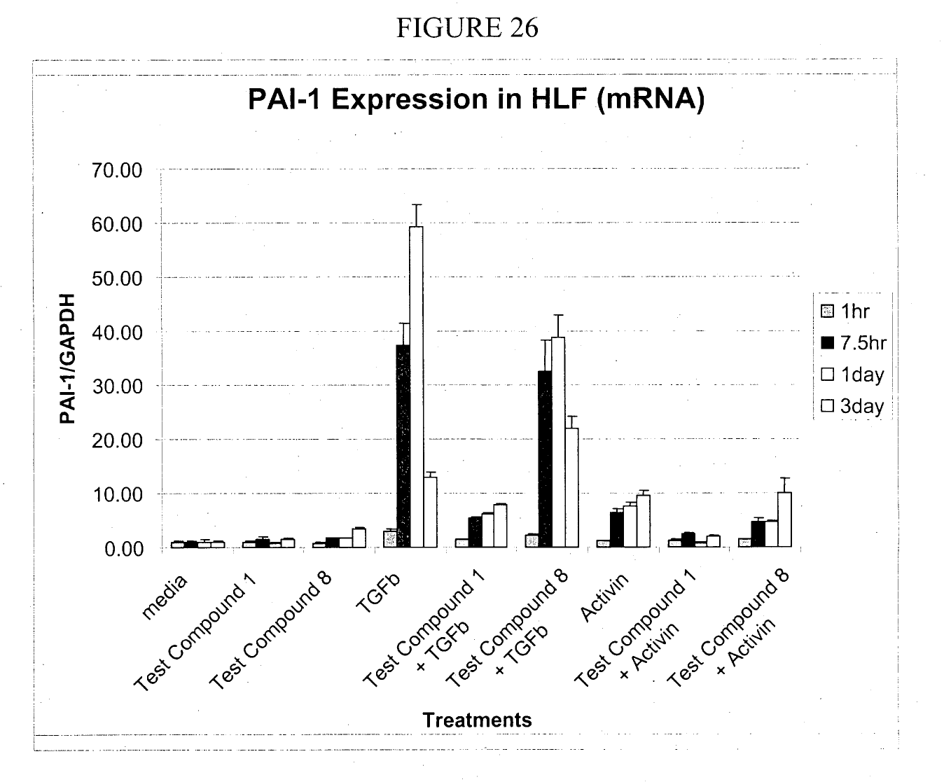

FIGS. 27A, 27B and 27C show glucocorticoid receptor mRNA expression regulated by TGF-β in HLF in the presence of a test compound at day 1, 2 and 3, respectively.

FIGS. 28A, 28B and 28C show Smad2 mRNA expression regulated TGF-β in HLF in the presence of a test compound at day 1, 2 and 3, respectively.

FIGS. 29A, 29B and 29C show Smad3 mRNA expression regulated TGF-β in HLF in the presence of a test compound at day 1, 2 and 3, respectively.

FIGS. 30A, 30B and 30C show Smad4 mRNA expression regulated TGF-β in HLF in the presence of a test compound at day 1, 2 and 3, respectively.

FIGS. 31A, 31B and 31C show Smad7 mRNA expression regulated TGF-β in HLF in the presence of a test compound at day 1, 2 and 3, respectively.

FIGS. 32A, 32B and 32C show CTGF mRNA expression regulated TGF-β in HLF in the presence of a test compound at day 1, 2 and 3, respectively.

FIGS. 33A, 33B and 33C show Fibronectin (FN) mRNA expression regulated TGF-β in HLF in the presence of a test compound at day 1, 2 and 3, respectively.

FIGS. 34A, 34B and 34C show Col 1 mRNA expression regulated TGF-β in HLF in the presence of a test compound at day 1, 2 and 3, respectively.

FIGS. 35A, 35B and 35C show Col 3 mRNA expression regulated TGF-β in HLF in the presence of a test compound at day 1, 2 and 3, respectively.

FIGS. 36A, 36B and 36C show PAI-1 mRNA expression regulated TGF-β in HLF in the presence of a test compound at day 1, 2 and 3, respectively.

FIGS. 37A, 37B and 37C show IL-6 mRNA expression regulated TGF-β in HLF in the presence of a test compound at day 1, 2 and 3, respectively.

FIGS. 38A, 38B and 38C show TGF-β activated kinase 1 (TAK1) mRNA expression regulated TGF-β in HLF in the presence of a test compound at day 1, 2 and 3, respectively.

FIGS. 39A, 39B and 39C show p38 alpha (p38a) mRNA expression regulated TGF-β in HLF in the presence of a test compound at day 1, 2 and 3, respectively.

FIGS. 40A and 40B show β-actin mRNA expression regulated TGF-β in HLF in the presence of a test compound at day 2 and 3, respectively.

FIGS. 41A and 41B show Cox1 mRNA expression regulated TGF-β in HLF in the presence of a test compound at day 2 and 3, respectively.

FIG. 42 shows Cox2 mRNA expression regulated TGF-β in -HLF in the presence of a test compound at day 3.

FIGS. 43A and 43B show I kappa-B kinase (iKKi) mRNA expression regulated TGF-β in HLF in the presence of a test compound at day 2 and 3, respectively.

FIG. 44 shows effects of dexamethasone (Dex) and/or a test compound on bleomycin induced change in body weight.

FIG. 45 shows effects of dexamethasone (Dex) and/or a test compound on bleomycin induced change in total hydroxyproline in the lung.

FIG. 46 shows effects of dexamethasone (Dex) and/or a test compound on bleomycin induced change in lung capacity.

### DETAILED DESCRIPTION OF THE PREFERRED EMBODIMENT

A. Definitions

As used herein, the terms “fibroproliferative disease,” “fibroproliferative disorder,” and “fibrotic disease” are used interchangeably and in the broadest sense, to describe conditions characterized by or associated with unwanted or excessive fibrosis and/or sclerosis, including consequences of or complications resulting from such fibrosis and/or sclerosis, and symptoms of such fibrosis and/or sclerosis and of such consequences or complications.

As used herein, any reference to “reversing the effect of TGF-β -mediated cell activation on the expression of a gene associated with fibrosis” means partial or complete reversal the effect of TGF-β-mediated cell activation of that gene, relative to a normal sample of the same cell or tissue type. If is emphasized that total reversal (i.e. total return to the normal expression level) is not required, although is advantageous, under this definition.

The terms “specifically binding,” “binds specifically,” “specific binding,” and grammatical equivalents thereof, are used to refer to binding to a unique epitope within the type I TGF-β receptor (TGFβ-R1). The binding must occur with an affinity to effectively inhibit TGF-β signaling through TGFβ-R1.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a test sample relative to its expression in a normal or control sample. For the purpose of this invention, “differential gene expression” is considered to be present when there is at least an about 2.5-fold, preferably at least about 4-fold, more preferably at least about 6-fold, most preferably at least about 10-fold difference between the expression of a given gene in normal and test samples.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. In the treatment of a fibroproliferative disease, a therapeutic agent may directly decrease the pathology of the disease, or render the disease more susceptible to treatment by other therapeutic agents.

The “pathology” of a fibroproliferative disease includes all phenomena that compromise the well-being of the patient. This includes, without limitation, unwanted or excessive fibrosis and/or sclerosis, release of various proteins associated with excessive fibrosis and/or sclerosis at abnormal levels, conditions characterized by or associated with unwanted or excessive fibrosis and/or sclerosis, including consequences of or complications resulting from such fibrosis and/or sclerosis, and symptoms of such fibrosis and/or sclerosis and such consequences or complications, etc.

The term “inhibitor” as used herein refers to a molecule, e.g. a nonpeptide small molecule, specifically binding to a TGFβ-R1 receptor having the ability to inhibit the biological function of a native TGF-β molecule. Accordingly, the term “inhibitor” is defined in the context of the biological role of TGF-β and its receptors.

The term “preferentially inhibit” as used herein means that the inhibitory effect on the target that is “preferentially inhibited” is significantly greater than on any other target. Thus, in the context of preferential inhibition of TGF-β-R1 kinase relative to the p38 kinase, the term means that the inhibitor inhibits biological activities, e.g. profibrotic activities, mediated by the TGF-β-R1 kinase significantly more than biological activities mediated by the p38 kinase. The difference in the degree of inhibition, in favor of the preferentially inhibited receptor, generally is at least about two-fold, more preferably at least about five-fold, even more preferably at least about ten-fold.

The term “mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

A “therapeutically effective amount”, in reference to the treatment of a fibrotic disease, e.g. when inhibitors of the present invention are used, refers to an amount capable of invoking one or more of the following effects: (1) inhibition (i.e., reduction, slowing down or complete stopping) of the development or progression of fibrosis and/or sclerosis; (2) inhibition (i.e., reduction, slowing down or complete stopping) of consequences of or complications resulting from such fibrosis and/or sclerosis; and (3) relief, to some extent, of one or more symptoms associated with the fibrosis and/or sclerosis, or symptoms of consequences of or complications resulting from such fibrosis and/or sclerosis.

As used herein, a “noninterfering substituent” is a substituent which leaves the ability of the compound as described in the formulas provided herein to inhibit TGF-β activity qualitatively intact. Thus, the substituent may alter the degree of inhibition. However, as long as the compound retains the ability to inhibit TGF-β activity, the substituent will be classified as “noninterfering.”

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when indicated, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O , S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety or “aryl” moiety refers to a monocyclic or fused bicyclic moiety such as phenyl or naphthyl; “heteroaromatic” also refers to monocyclic or fused bicyclic ring systems containing one ore more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C or 1-8C, or the hetero forms thereof. These carbon chains may also include a carbonyl group, thus making them able to provide substituents as an acyl or heteroacyl moiety.

B. Modes of Carrying out the Invention

TGF-β plays a central role in fibrosis. The present invention provides TGF-β inhibitors which, via binding to TGFβ-R1, find utility in the treatment of fibroproliferative diseases. As discussed before, the biological activities of TGF-β are mediated by two distinct types of receptors designated type I and type II (Derynck and Feng, Biochim. Biophys. Acta 1333:F105-F150 (1997); Massague, Annu. Rev. Biochem., 67:753-91 (1998)). Both receptors are serine-threonine kinases. Upon binding of TGF-β to the type II receptor, the type II receptor phosphorylates the type I receptor, which is activated and is, in turn, responsible for intracellular signaling. In addition, TGF-β has a non-serine-theronine kinase receptor, termed type III receptor, which is believed to facilitate or modulate signaling through the type I/II receptor pair (Lopez-Casillas et al., Cell 73:996-1005 (1993)).

The present invention is based on the surprising finding that certain quinazoline derivatives specifically binding to the type I TGF-β receptor (TGFβ-R1) can effectively block fibrosis mediated by signaling through this complex receptor system, and through downstream signaling pathways.

In a preferred embodiment, the inhibitors of the present invention selectively inhibit biological responses mediated by the type I receptor, in particular matrix production, without affecting the type II receptor-mediated cell proliferation.

In another preferred embodiment, the compounds of the present invention preferentially inhibit TGF-β R1 kinase relative to p38 kinase.

Compounds of the Invention

The inhibitors of the present invention typically are small organic molecules (non-peptide small molecules), generally less than about 1,000 daltons in size. Preferred non-peptide small molecules have molecular weights of less than about 750, daltons, more preferably less than about 500 daltons, and even more preferably less than about 300 daltons. Similar compounds are disclosed in WO 00/12497, which is expressly incorporated herein by reference.

In a preferred embodiment, the compounds are of the formula

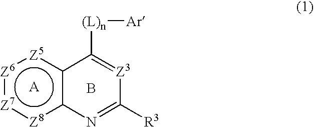

or the pharmaceutically acceptable salts thereof

wherein R³ is a noninterfering substituent;

each Z is CR² or N, wherein no more than two Z positions in ring A are N, and wherein two adjacent Z positions in ring A cannot be N;

each R² is independently a noninterfering substituent;

L is a linker;

n is 0 or 1; and

Ar′ is the residue of a cyclic aliphatic, cyclic heteroaliphatic, aromatic or heteroaromatic moiety optionally substituted with 1-3 noninterfering substituents.

In a more preferred embodiment, the small organic molecules herein are derivatives of quinazoline and related compounds containing mandatory substituents at positions corresponding to the 2- and 4-positions of quinazoline. In general, a quinazoline nucleus is preferred, although alternatives within the scope of the invention are also illustrated below. Preferred embodiments for Z³ are N and CH; preferred embodiments for Z⁵-Z⁸ are CR². However, each of Z⁵-Z⁸ can also be N, with the proviso noted above. Thus, with respect to the basic quinazoline type ring system, preferred embodiments include quinazoline per se, and embodiments wherein all of Z⁵-Z⁸ as well as Z³ are either N or CH. Also preferred are those embodiments wherein Z³ is N, and either Z⁵ or Z⁸ or both Z⁵ and Z⁸ are N and Z⁶ and Z⁷ are CH or CR². Where R² is other than H, it is preferred that CR² occur at positions 6 and/or 7. Thus, by way of example, quinazoline derivatives within the scope of the invention include compounds comprising a quinazoline nucleus, having an aromatic ring attached in position 2 as a non-interfering substituent (R³), which may be further substituted.

With respect to the substituent at the positions corresponding to the 4-position of quinazoline, LAr′, L is present or absent and is a linker which spaces the substituent Ar′ from ring B at a distance of 2-8Å, preferably 2-6Å, more preferably 2-4Å. The distance is measured from the ring carbon in ring B to which one valence of L is attached to the atom of the Ar′ cyclic moiety to which the other valence of the linker is attached. The Ar′ moiety may also be coupled directly to ring B (i.e., when n is 0). Typical, but nonlimiting, embodiments of L are of the formula S(CR²₂)ₘ, —NR¹SO₂(CR²₂)₁, NR¹(CR²₂)ₘ, NR¹CO(CR²₂)₁, O(CR²₂)ₘ, OCO(CR²₂)₁, and

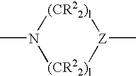

wherein Z is N or CH and wherein m is 0-4 and 1 is 0-3, preferably 1-3 and 1-2, respectively. L preferably provides —NR¹— coupled directly to ring B. A preferred embodiment of R¹ is H, but R¹ may also be acyl, alkyl, arylacyl or arylalkyl where the aryl moiety may be substituted by 1-3 groups such as alkyl, alkenyl, alkynyl, acyl, aryl, alkylaryl, aroyl, N-aryl, NH-alkylaryl, NH-aroyl, halo, OR, NR₂, SR, —SOR, —NRSOR, —NRSO₂R, —SO₂R, —OCOR, —NRCOR, —NRCONR₂, —NRCOOR, —OCONR₂, —RCO, —COOR, —SO₃R, —CONR₂, SO₂NR₂, CN, CF₃, and NO₂, wherein each R is independently H or alkyl (1-4C), preferably the substituents are alkyl (1-6C), OR, SR or NR₂ wherein R is H or lower alkyl (1-4C). More preferably, R¹ is H or alkyl (1-6C). Any aryl groups contained in the substituents may further be substituted by for example alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, CN, CF₃, or NO₂, wherein each R is independently H or alkyl (1-4C).

Ar′ is aryl, heteroaryl, including 6-5 fused heteroaryl, cycloaliphatic or cycloheteroaliphatic. Preferably Ar′ is phenyl, 2-, 3- or 4-pyridyl, indolyl, 2- or 4-pyrimidyl, benzimidazolyl, indolyl, preferably each optionally substituted with a group selected from the group consisting of optionally substituted alkyl, alkenyl, alkynyl, aryl, N-aryl, NH-aroyl, halo, OR, NR₂, SR, —OOCR, —NROCR, RCO, —COOR, —CONR₂, SO₂NR₂, CN, CF₃, and NO₂, wherein each R is independently H or alkyl (1-4C).

Ar′ is more preferably indolyl, 6-pyrimidyl, 3- or 4-pyridyl, or optionally substituted phenyl.

For embodiments wherein Ar′ is optionally substituted phenyl, substituents include, without limitation, alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). Preferred substituents include halo, OR, SR, and NR₂ wherein R is H or methyl or ethyl. These substituents may occupy all five positions of the phenyl ring, preferably 1-2 positions, preferably one position. Embodiments of Ar′ include substituted or unsubstituted phenyl, 2-, 3-, or 4-pyridyl, 2-, 4- or 6-pyrimidyl, indolyl, isoquinolyl, quinolyl, benzimidazolyl, benzotriazolyl, benzothiazolyl, benzofuranyl, pyridyl, thienyl, furyl, pyrrolyl, thiazolyl, oxazolyl, imidazolyl, and morpholinyl. Particularly preferred as an embodiment of Ar′ is 3- or 4-pyridyl, especially 4-pyridyl in unsubstituted form.

Any of the aryl moieties, especially the phenyl moieties, may also comprise two substituents which, when taken together, form a 5-7 membered carbocyclic or heterocyclic aliphatic ring.

Thus, preferred embodiments of the substituents at the position of ring B corresponding to 4-position of the quinazoline include 2-(4-pyridyl)ethylamino; 4-pyridylamino; 3-pyridylamino; 2-pyridylamino; 4-indolylamino; 5-indolylamino; 3-methoxyanilinyl; 2-(2,5-difluorophenyl)ethylamino-, and the like.

R³is generally a hydrocarbyl residue (1-20C) containing 0-5 heteroatoms selected from O, S and N. Preferably R³ is alkyl, aryl, arylalkyl, heteroalkyl, heteroaryl, or heteroarylalkyl, each unsubstituted or substituted with 1-3 substituents. The substituents are independently selected from a group that includes halo, OR, NR₂, SR, —SOR, —SO₂R, —OCOR, —NRCOR, —NRCONR₂, —NRCOOR, —OCONR₂, RCO, —COOR, —SO₃R, NRSOR, NRSO₂R, —CONR₂, SO₂NR₂, CN, CF₃, and NO₂, wherein each R is independently H or alkyl (1-4C) and with respect to any aryl or heteroaryl moiety, said group further including alkyl (1-6C) or alkenyl or alkynyl. Preferred embodiments of R³ (the substituent at position corresponding to the 2-position of the quinazoline) comprise a phenyl moiety optionally substituted with 1-2 substituents preferably halo, alkyl (1-6C), OR, NR₂, and SR wherein R is as defined above. Thus, preferred substituents at the 2-position of the quinazoline include phenyl, 2-halophenyl, e.g., 2-bromophenyl, 2-chlorophenyl, 2-fluorophenyl; 2-alkyl-phenyl, e.g., 2-methylphenyl, 2-ethylphenyl; 4-halophenyl, e.g., 4-bromophenyl, 4-chlorophenyl, 4-fluorophenyl; 5-halophenyl, e.g. 5-bromophenyl, 5-chlorophenyl, 5-fluorophenyl; 2,4- or 2,5-halophenyl, wherein the halo substituents at different positions may be identical or different, e.g. 2-fluoro-4-chlorophenyl; 2-bromo-4-chlorophenyl; 2-fluoro-5-chlorophenyl; 2-chloro-5-fluorophenyl, and the like. Other preferred embodiments of R³ comprise a cyclopentyl or cyclohexyl moiety.

As noted above, R² is a noninterfering substituent. As set forth above, a “noninterfering substituent” is one whose presence does not substantially destroy the TGF-β inhibiting ability of the compound of formula (1).

Each R² is also independently a hydrocarbyl residue (1-20C) containing 0-5 heteroatoms selected from O, S and N. Preferably, R² is independently H, alkyl, alkenyl, alkynyl, acyl or hetero-forms thereof or is aryl, arylalkyl, heteroalkyl, heteroaryl, or heteroarylalkyl, each unsubstituted or substituted with 1-3 substituents selected independently from the group consisting of alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C). The aryl or aroyl groups on said substituents may be further substituted by, for example, alkyl, alkenyl, alkynyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). More preferably the substituents on R² are selected from R⁴, halo, OR⁴, NR⁴², SR⁴, —OOCR⁴, —NROCR⁴, —COOR⁴, R⁴CO, —CONR⁴₂, —SO₂NR⁴₂, CN, CF₃, and NO₂, wherein each R⁴ is independently H, or optionally substituted alkyl (1-6C), or optionally substituted arylalkyl (7-12C) and wherein two R⁴ or two substituents on said alkyl or arylalkyl taken together may form a fused aliphatic ring of 5-7 members.

R₂ may also, itself, be selected from the group consisting of halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO ₂, wherein each R is independently H or alkyl (1-4C).

More preferred substituents represented by R² are those as set forth with regard to the phenyl moieties contained in Ar′ or R³ as set forth above. Two adjacent CR² taken together may form a carbocyclic or heterocyclic fused aliphatic ring of 5-7 atoms. Preferred R² substituents are of the formula R⁴, —OR⁴, SR⁴ or R⁴NH—, especially R⁴NH—, wherein R⁴ defined as above. Particularly preferred are instances wherein R⁴ is substituted arylalkyl. Specific representatives of the compounds of formula (1) are shown in Tables 1-3 below. All compounds listed in Table 1 have a quinazoline ring system (Z³ is N), where the A ring is unsubstituted (Z⁵-Z⁸ represent CH). The substituents of the B ring are listed in the table.

TABLE 1

| Compound No. | L | Ar′ | R³ |
|---|---|---|---|
| 1 | NH | 4-pyridyl | 2-chlorophenyl |
| 2 | NH | 4-pyridyl | 2,6-dichlorophenyl |
| 3 | NH | 4-pyridyl | 2-methylphenyl |
| 4 | NH | 4-pyridyl | 2-bromophenyl |
| 5 | NH | 4-pyridyl | 2-fluorophenyl |
| 6 | NH | 4-pyridyl | 2,6-difluorophenyl |
| 7 | NH | 4-pyridyl | phenyl |
| 8 | NH | 4-pyridyl | 4-fluorophenyl |
| 9 | NH | 4-pyridyl | 4-methoxyphenyl |
| 10 | NH | 4-pyridyl | 3-fluorophenyl |
| 11* | N* | 4-pyridyl | phenyl |
| 12† | N† | 4-pyridyl | phenyl |
| 13 | NHCH₂ | 4-pyridyl | phenyl |
| 14 | NHCH₂ | 4-pyridyl | 4-chlorophenyl |
| 15 | NH | 3-pyridyl | phenyl |
| 16 | NHCH₂ | 2-pyridyl | phenyl |
| 17 | NHCH₂ | 3-pyridyl | phenyl |
| 18 | NHCH₂ | 2-pyridyl | phenyl |
| 19 | NHCH₂CH₂ | 2-pyridyl | phenyl |
| 20 | NH | 6-pyrimidinyl | phenyl |
| 21 | NH | 2-pyrimidinyl | phenyl |
| 22 | NH | phenyl | phenyl |
| 23 | NHCH₂ | phenyl | 3-chlorophenyl |
| 24 | NH | 3-hydroxyphenyl | phenyl |
| 25 | NH | 2-hydroxyphenyl | phenyl |
| 26 | NH | 4-hydroxyphenyl | phenyl |
| 27 | NH | 4-indolyl | phenyl |
| 28 | NH | 5-indolyl | phenyl |
| 29 | NH | 4-methoxyphenyl | phenyl |
| 30 | NH | 3-methoxyphenyl | phenyl |
| 31 | NH | 2-methoxyphenyl | phenyl |
| 32 | NH | 4-(2-hydroxyethyl)phenyl | phenyl |
| 33 | NH | 3-cyanophenyl | phenyl |
| 34 | NHCH₂ | 2,5-difluorophenyl | phenyl |
| 35 | NH | 4-(2-butyl)phenyl | phenyl |
| 36 | NHCH₂ | 4-dimethylaminophenyl | phenyl |
| 37 | NH | 4-pyridyl | cyclopentyl |
| 38 | NH | 2-pyridyl | phenyl |
| 39 | NHCH₂ | 3-pyridyl | phenyl |
| 40 | NH | 4-pyrimidyl | phenyl |
| 41‡ | N‡ | 4-pyridyl | phenyl |
| 42 | NH | p-aminomethylphenyl | phenyl |
| 43 | NHCH₂ | 4-aminophenyl | phenyl |
| 44 | NH | 4-pyridyl | 3-chlorophenyl |
| 45 | NH | phenyl | 4-pyridyl |
| 46 | NH | 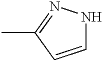 | phenyl |
| 47 | NH | 4-pyridyl | t-butyl |
| 48 | NH | 2-benzylamino-3-pyridyl | phenyl |
| 49 | NH | 2-benzylamino-4-pyridyl | phenyl |
| 50 | NH | 3-benzyloxyphenyl | phenyl |
| 51 | NH | 4-pyridyl | 3-aminophenyl |
| 52 | NH | 4-pyridyl | 4-pyridyl |
| 53 | NH | 4-pyridyl | 2-naphthyl |
| 54 | 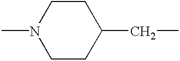 | 4-pyridyl | phenyl |
| 55 | 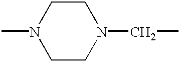 | phenyl | phenyl |
| 56 | 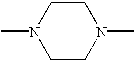 | 2-pyridyl | phenyl |
| 57 | NHCH₂CH₂ | 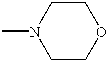 | phenyl |
| 58 | not present | 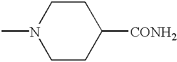 | phenyl |
| 59 | not present | 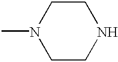 | phenyl |
| 60 | NH | 4-pyridyl | cyclopropyl |
| 61 | NH | 4-pyridyl | 2-trifluoromethylphenyl |
| 62 | NH | 4-aminophenyl | phenyl |
| 63 | NH | 4-pyridyl | cyclohexyl |
| 64 | NH | 3-methoxyphenyl | 2-fluorophenyl |
| 65 | NH | 4-methoxyphenyl | 2-fluorophenyl |
| 66 | NH | 4-pyrimidinyl | 2-fluorophenyl |
| 67 | NH | 3-amino-4-pyridyl | phenyl |
| 68 | NH | 4-pyridyl | 2-benzylaminophenyl |
| 69 | NH | 2-benzylaminophenyl | phenyl |
| 70 | NH | 2-benzylaminophenyl | 4-cyanophenyl |
| 71 | NH | 3′-cyano-2-benzylaminophenyl | phenyl |

*R¹ = 2-propyl
†R¹ = 4-methoxyphenyl
‡R¹ = 4-methoxybenzyl

The compounds in Table 2 contain modifications of the quinazoline nucleus as shown. All of the compounds in Table 2 are embodiments of formula (1) wherein Z³ is N and Z⁶ and Z⁷ represent CH. In all cases the linker, L, is present and is NH.

TABLE 2

| Compound No. | Z⁵ | Z⁸ | Ar′ | R³ |
|---|---|---|---|---|
| 72 | CH | N | 4-pyridyl | 2-fluorophenyl |
| 73 | CH | N | 4-pyridyl | 2-chlorophenyl |
| 74 | CH | N | 4-pyridyl | 5-chloro-2- |
| | | | | fluorphenyl |
| 75 | CH | N | 4-(3-methyl)-pyridyl | 5-chloro-2- |
| | | | | fluorphenyl |
| 76 | CH | N | 4-pyridyl | Phenyl |
| 77 | N | N | 4-pyridyl | phenyl |
| 78 | N | CH | 4-pyridyl | Phenyl |
| 79 | N | N | 4-pyridyl | 5-chloro-2- |
| | | | | fluorphenyl |
| 80 | N | N | 4-(3-methyl)-pyridyl | 5-chloro-2- |
| | | | | fluorphenyl |

Additional compounds were prepared wherein ring A contains CR² at Z⁶ or Z⁷ where R² is not H. These compounds, which are all quinazoline derivatives, wherein L is NH and Ar′ is 4-pyridyl, are shown in Table 3.

TABLE 3

| Compound No. | R³ | CR² as noted |
|---|---|---|
| 81 | 2-chlorophenyl | 6,7-dimethoxy |
| 82 | 2-fluorophenyl | 6-nitro |
| 83 | 2-fluorophenyl | 6-amino |
| 84 | 2-fluorophenyl | 7-amino |
| 85 | 2-fluorophenyl | 6-(3-methoxybenzylamino) |
| 86 | 2-fluorophenyl | 6-(4-methoxybenzylamino) |
| 87 | 2-fluorophenyl | 6-(2-isobutylamino) |
| 88 | 2-fluorophenyl | 6-(4- |
| | | methylmercaptobenzylamino) |
| 89 | 2-fluorophenyl | 6-(4-methoxybenz amino) |
| 90 | 4-fluorophenyl | 7-amino |
| 91 | 4-fluorophenyl | 7-(3-methoxybenzylamino) |

Structures representative of quinazoline derivatives are shown below in Table 4.

TABLE 4

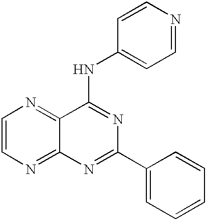

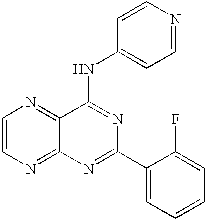

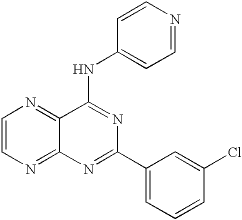

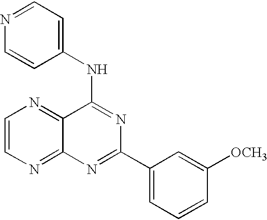

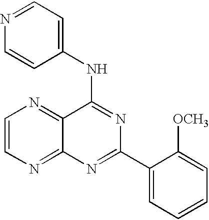

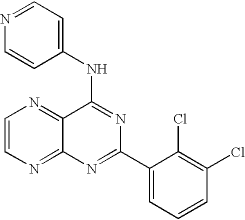

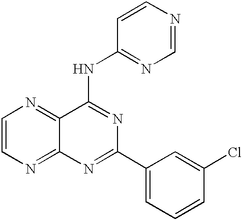

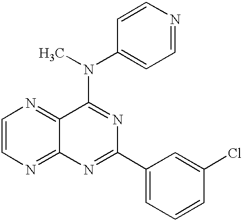

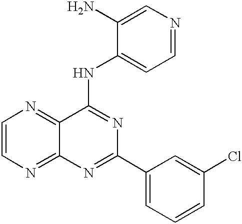

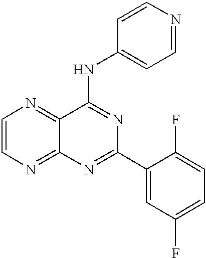

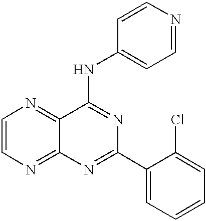

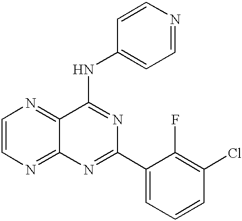

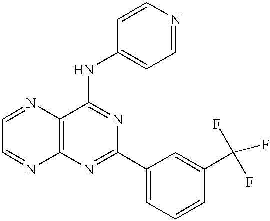

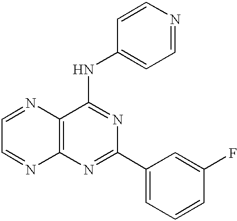

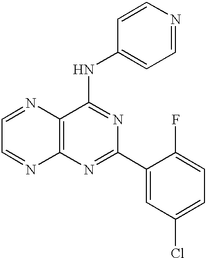

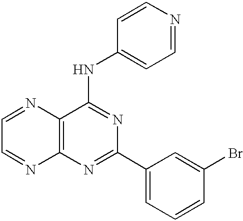

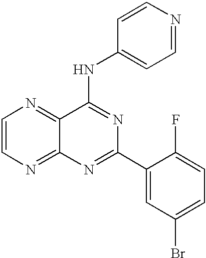

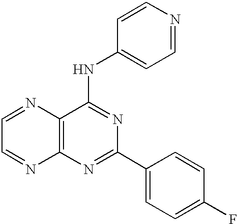

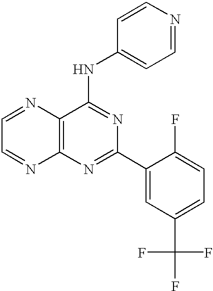

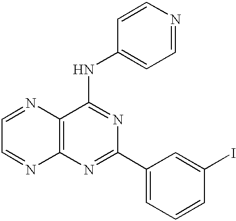

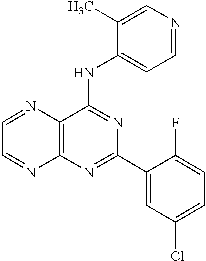

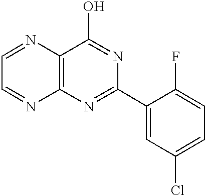

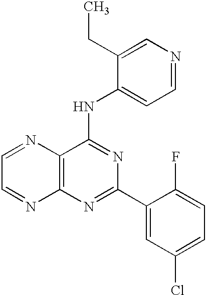

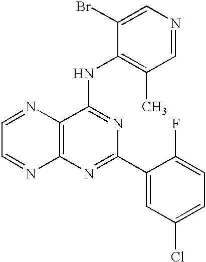

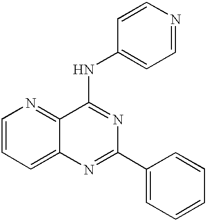

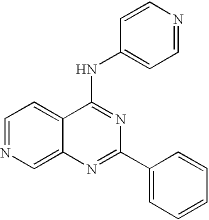

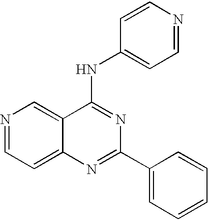

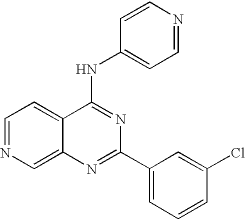

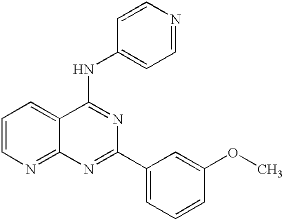

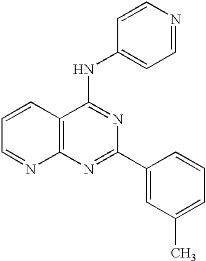

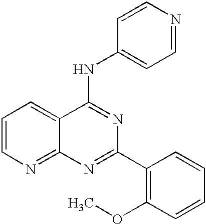

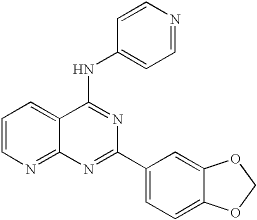

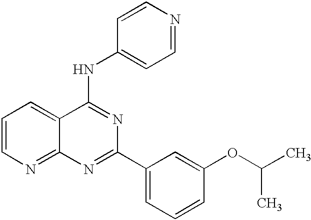

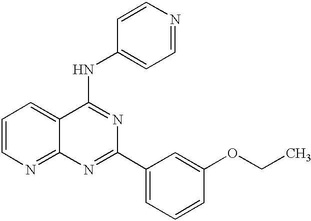

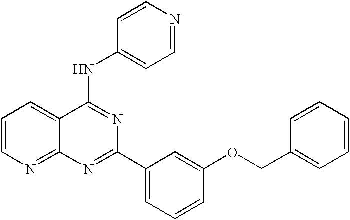

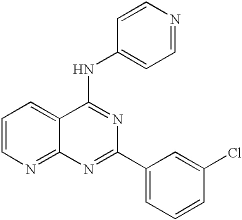

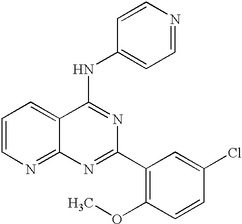

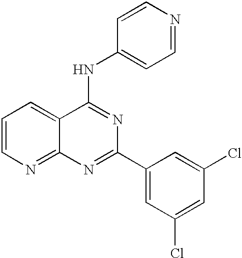

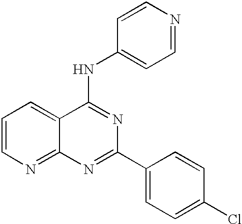

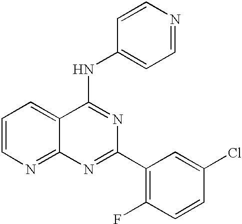

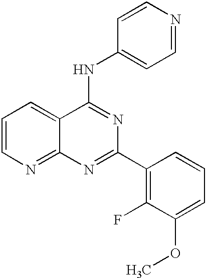

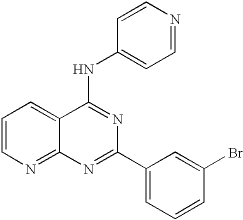

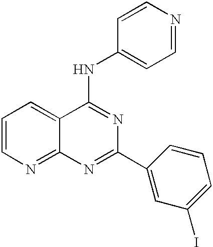

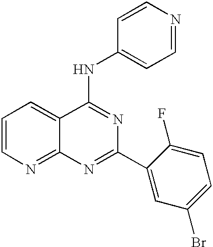

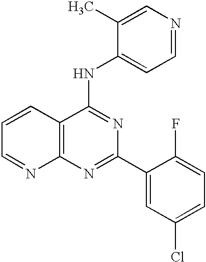

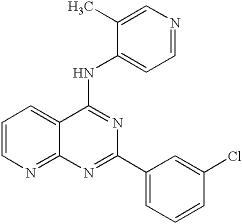

Although the invention is illustrated with reference to certain quinazoline derivatives, it is not so limited. Inhibitors of the present invention include compounds having a non-quinazoline, such as, a pyridine, such as, a pyridine, pyrimidine nucleus carrying substituents like those discussed above with respect to the quinazoline derivatives.

For example, in another embodiment, the compounds are of the formula

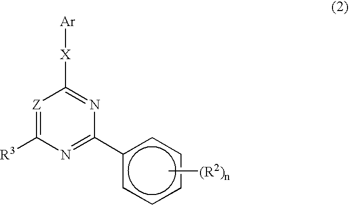

and the pharmaceutically acceptable salts and prodrug forms thereof; wherein

Ar represents an optionally substituted aromatic or optionally substituted heteroaromatic moiety containing 5-12 ring members wherein said heteroaromatic moiety contains one or more O, S, and/or N;

X is NR¹, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5.

In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

In preferred embodiments, Ar represents an optionally substituted aromatic or optionally substituted heteroaromatic moiety containing 5-9 ring members wherein said heteroaromatic moiety contains one or more N; or

R¹ is H, alkyl (1-8C), alkenyl (2-8C), or alkynyl (2-8C); or

Z represents N or CR⁴; wherein

R⁴ is H, alkyl (1-10C), alkenyl (2-10C), or alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or alkyl (1-10C) or a halo or heteroatom-containing form of, said alkyl, each of which may optionally be substituted. Preferably R⁴ is H, alkyl (1-10C), OR, SR or NR₂ wherein R is H or alkyl (1-10C) or is O-aryl; or

R³ is defined in the same manner as R⁴ and preferred forms are similar, but R³ is independently embodied; or

each R² is independently alkyl (1-8C), alkenyl (2-8C), alkynyl (2-8C), acyl (1-8C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —NRSO₂R₂, —SO₂R, —OCOR, —OSO₃R, —NRCOR, —NRCONR₂, —NRCOOR, —OCONR₂, —COOR, —SO₃R, —CONR₂, SO₂NR₂, —CN, —CF₃, or —NO₂, wherein each R is independently H or lower alkyl (1 -4C). Preferably R² is halo, alkyl (1-6C), OR, SR or NR₂ wherein R is H or lower alkyl (1-4C), more preferably halo; or

n is 0-3.

The optional substituents on the aromatic or heteroaromatic moiety represented by Ar include alkyl (1-10C), alkenyl (2-10C), alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, -S0₃R, —CONR₂, —SO₂NR₂, —CN, —CF₃, and/or NO₂, wherein each R is independently H or lower alkyl (1-4C). Preferred substituents include alkyl, OR, NR₂, O-alkylaryl and NH-alkylaryl.

Because tautomers are theoretically possible, phthalimido is also considered aromatic, and phthalimido-substituted alkyl and phthalimido-substituted alkoxy are preferred embodiments of R³ and R⁴.

In general, any alkyl, alkenyl, alkynyl, acyl, or aryl group contained in a substituent may itself optionally be substituted by additional substituents. The nature of these substituents is similar to those recited with regard to the primary substituents themselves. Thus, where an embodiment of, for example, R⁴ is alkyl, this alkyl may optionally be substituted by the remaining substituents listed as embodiments for R⁴ where this makes chemical sense, and where this does not undermine the size limit of alkyl per se; e.g., alkyl substituted by alkyl or by alkenyl would simply extend the upper limit of carbon atoms for these embodiments. However, alkyl substituted by aryl, amino, alkoxy, and the like would be included within the scope of the invention. The features of the compounds are defined by formula (2) and the nature of the substituents is less important as long as the substituents do not interfere with the stated biological activity of this basic structure.

Non-interfering substituents embodied by R², R³ and R⁴, include, but are not limited to, alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, wherein each R is independently H or alkyl (1-8C), —CN, —CF₃, and NO₂, and like substituents. R³ and R⁴ can also be H. Preferred embodiments for R³ and R⁴ are H, alkyl (1-10C) or a heteroatom-containing form thereof, each optionally substituted, especially (1-4C) alkyl; alkoxy (1-8C), acylamido, aryloxy, arylalkyloxy, especially wherein the aryl group is a phthalimido group, and alkyl or arylalkyl amine. Preferred embodiments of R² include lower alkyl, alkoxy, and halo, preferably halo. Halo, as defined herein includes fluoro, chloro, bromo and iodo. Fluoro and chloro are preferred.

Preferably, R¹ is H or lower alkyl (1-4C), more preferably H.

Preferably Ar is optionally substituted phenyl, 2-, 3- or 4-pyridyl, indolyl, 2- or 4-pyrimidyl, pyridazinyl, benzotriazol or benzimidazolyl. More preferably Ar is phenyl, pyridyl, or pyrimidyl. Each of these embodiments may optionally be substituted with a group such as alkyl, alkenyl, alkynyl, aryl, O-aryl, O-alkylaryl, O-aroyl, NR-aryl, N-alkylaryl, NR-aroyl, halo, OR, NR₂, SR, —OOCR, —NROCR, RCO, —COOR, —CONR₂, and/or SO₂NR₂, wherein each R is independently H or alkyl (1-8C), and/or by —CN, —CF₃, and/or NO₂. Alkyl, alkenyl, alkynyl and aryl portions of these may be further substituted by similar substituents.

Preferred substituents on Ar include alkyl, alkenyl, alkynyl, halo, OR, SR, NR₂ wherein R is H or alkyl (1-4C); and/or arylamino, arylalkylamino, including alkylamino which is substituted by more than one aryl. As stated above, any aryl or alkyl group included within a substituent may itself be substituted similarly. These substituents may occupy all available positions of the ring, preferably 1-2 positions, or more preferably only one position.

Any of the aryl moieties, including those depicted in formula (2) especially the phenyl moieties, may also comprise two substituents which, when taken together, form a 5-7 membered carbocyclic or heterocyclic aliphatic ring. Similarly, R⁴ may be bridged to R³ to obtain a 5-7 membered carbocyclic or heterocyclic ring.

Structures representative of pyrimidine derivatives are shown below in Table 5.

TABLE 5

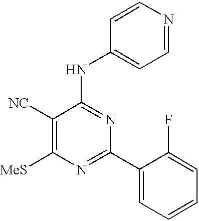

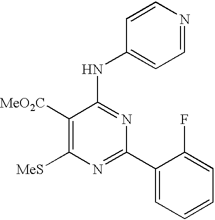

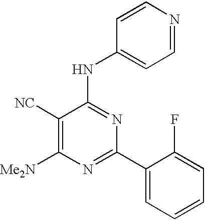

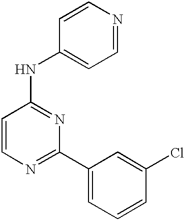

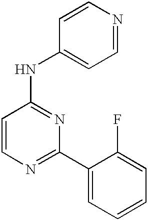

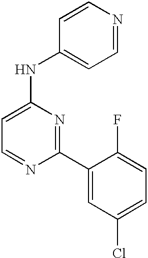

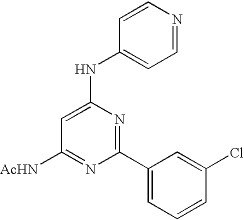

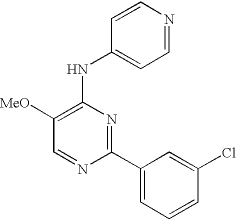

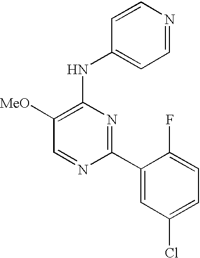

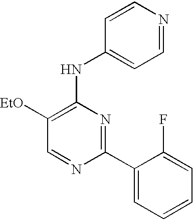

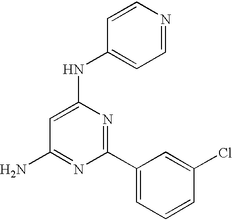

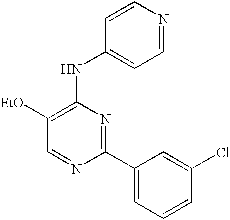

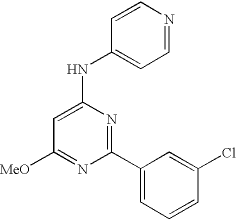

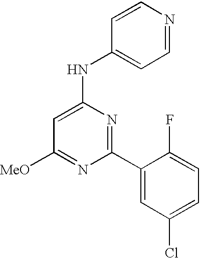

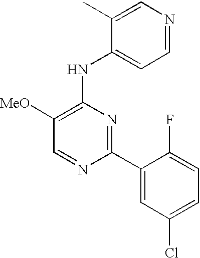

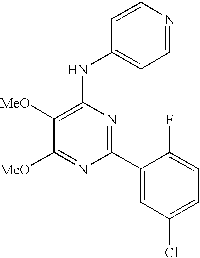

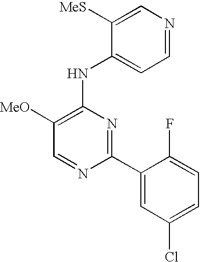

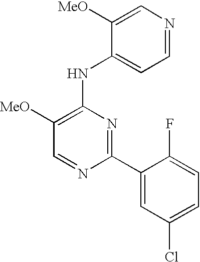

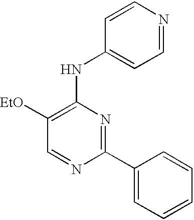

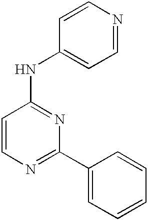

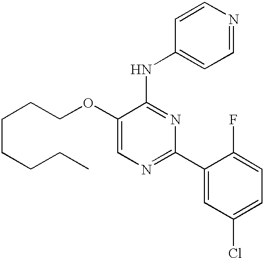

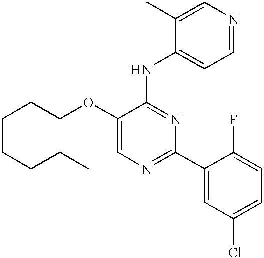

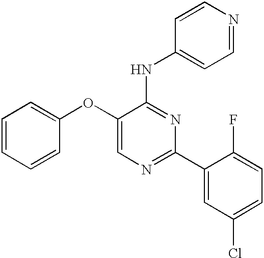

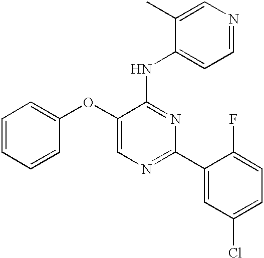

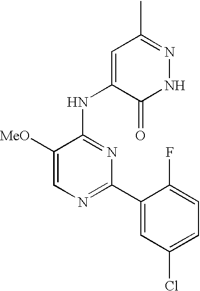

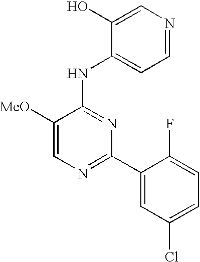

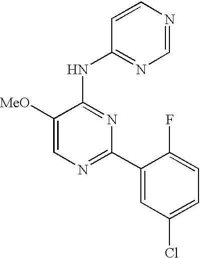

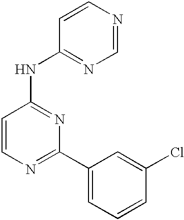

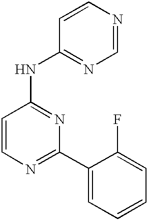

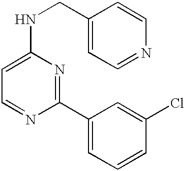

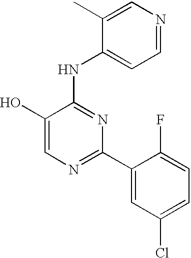

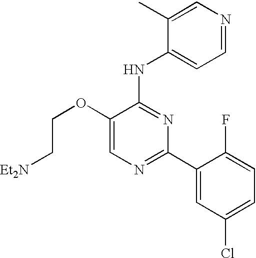

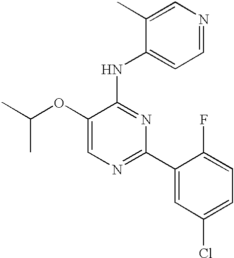

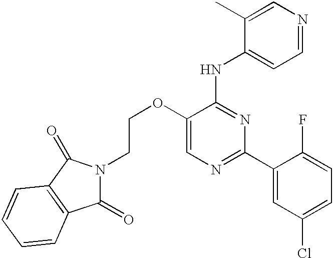

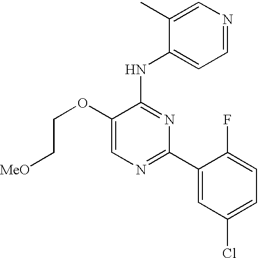

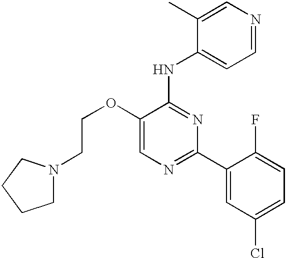

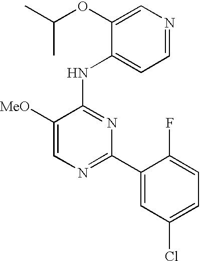

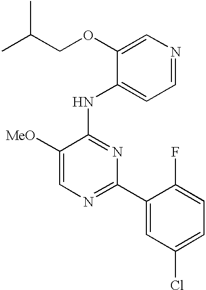

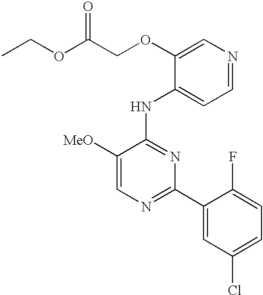

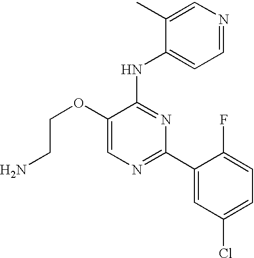

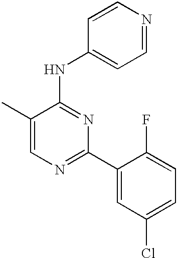

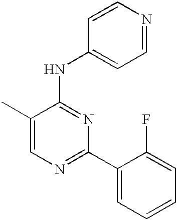

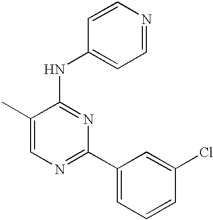

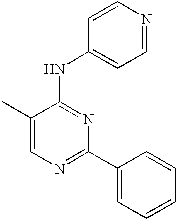

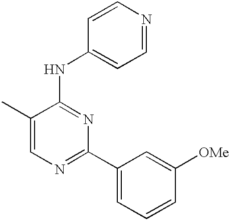

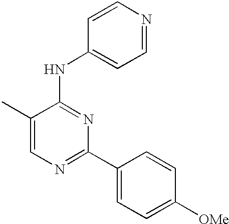

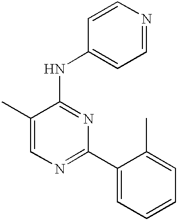

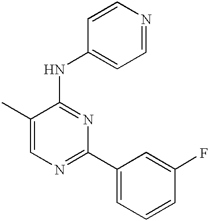

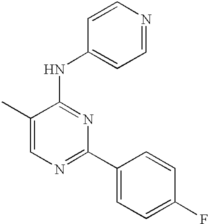

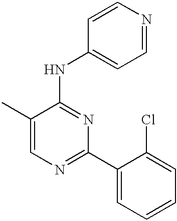

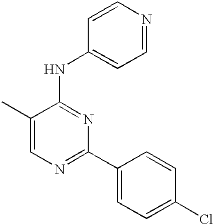

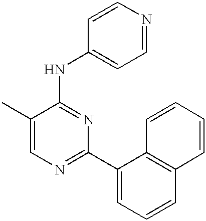

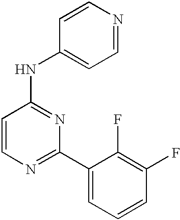

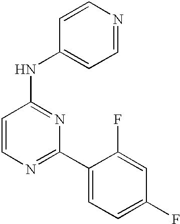

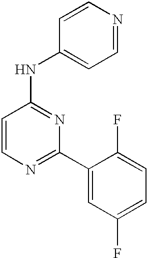

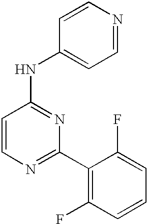

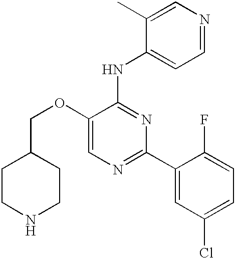

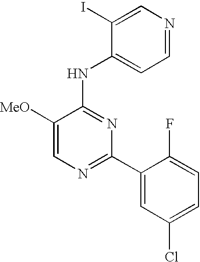

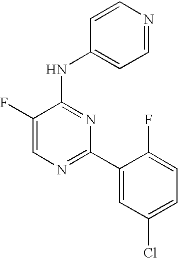

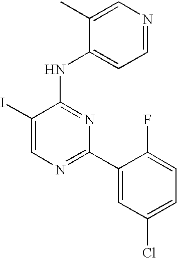

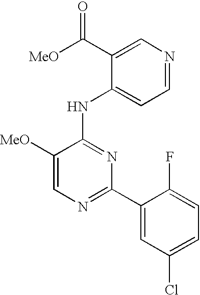

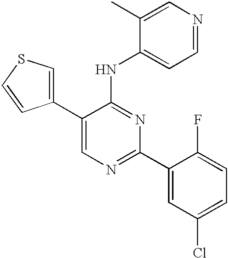

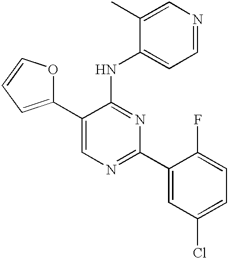

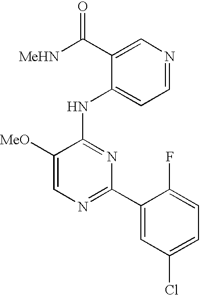

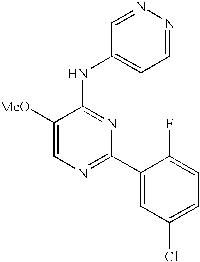

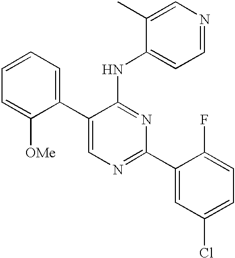

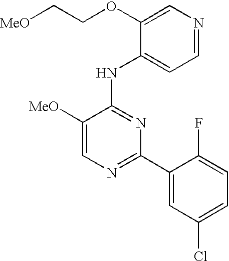

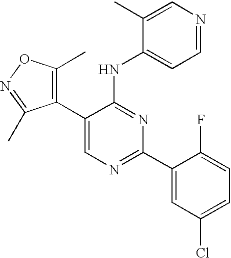

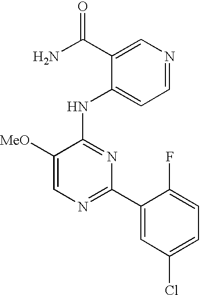

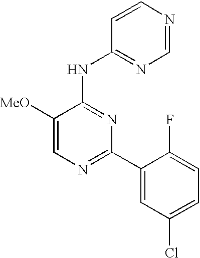

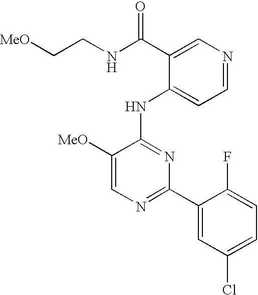

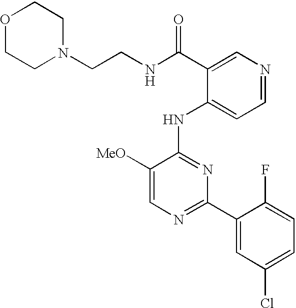

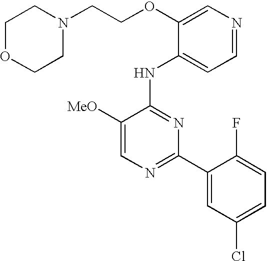

The compounds of the formula (1) and formula (2), may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1) or formula (2), the compound may also be supplied as a salt with a pharmaceutically acceptable cation.

The compounds of formula (1) and formula (2) may also be supplied in the form of a “prodrug” which is designed to release the compound of formula (1) or formula (2) when administered to a subject. Prodrug formed designs are well known in the art, and depend on the substituents contained in the compound of formula (1) or formula (2). For example, a substituent containing sulfhydryl could be coupled to a carrier which renders the compound biologically inactive until removed by endogenous enzymes or, for example, by enzymes targeted to a particular receptor or location in the subject.

In the event that any of the substituents of formula (2) contain chiral centers, as some, indeed, do, the compounds of formula (2) include all stereoisomeric forms thereof, both as isolated stereoisomers and mixtures of these stereoisomeric forms.

Synthesis of the Compounds of the Invention

The compounds of the formula (1) may be synthesized from the corresponding 4-halo-2-phenyl quinazoline as described in Reaction Scheme 1; which may be obtained from the corresponding 4-hydroxyquinazoline as shown in Reaction Scheme 2. Alternatively, the compounds can be prepared using anthranylamide as a starting material and benzoylating the amino group followed by cyclization to obtain the intermediate 2-phenyl-4-hydroxy quinazoline as shown in Reaction Scheme 3. Reaction Schemes 4-6 are similar to Reaction Scheme 3 except that an appropriate pyridine or 1,4-pyrimidine nucleus, substituted with a carboxamide residue and an adjacent amino residue, is substituted for the anthranylimide. The compounds of the invention wherein R¹ is H can be further derivatized to comprise other embodiments of R¹ as shown in Reaction Scheme 7.

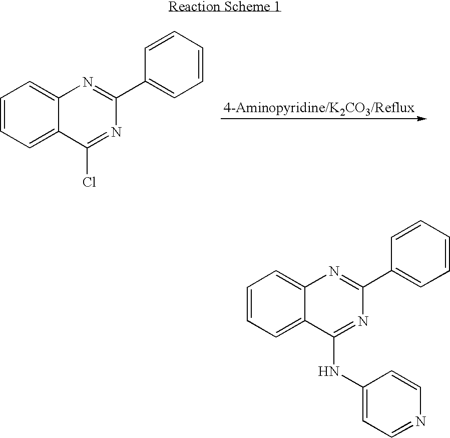

Reaction Scheme 1 is illustrative of the simple conversion of a halogenated quinazoline to compounds of the invention. Of course, the phenyl of the illustration at position 2 may be generalized as R³ and the 4-pyridylamino at position 2 can be generalized to Ar′-L or Ar′—.

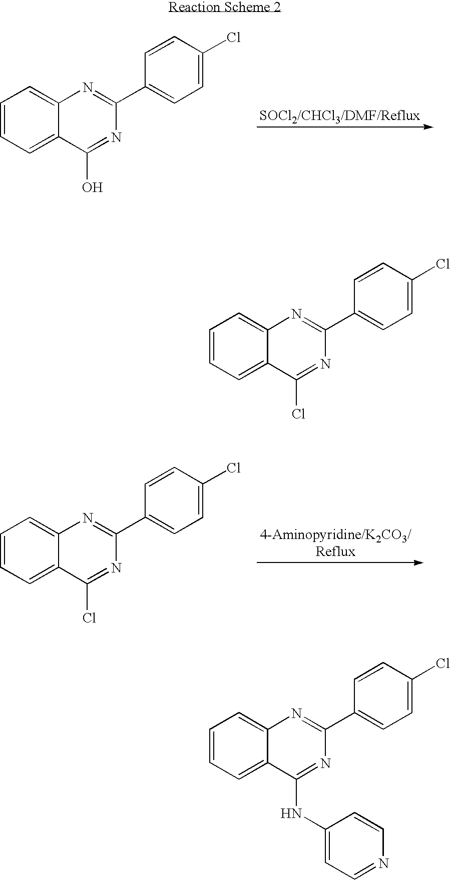

Reaction Scheme 2 can, of course, be generalized in the same manner as set forth for Reaction Scheme 1.

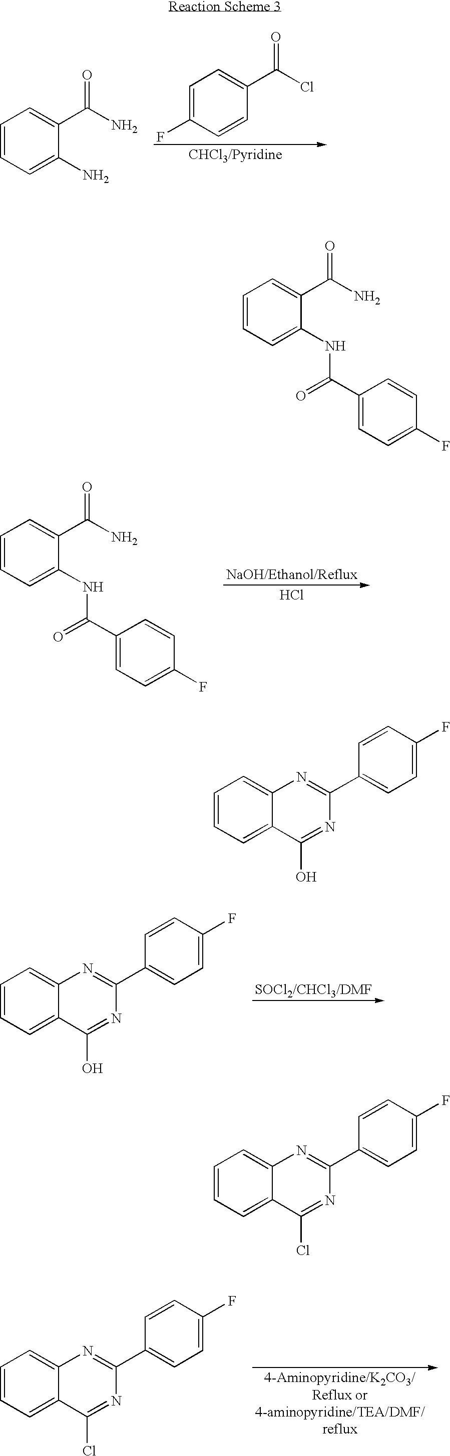

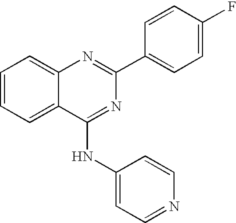

Again, Reaction Scheme 3 can be generalized by substituting the corresponding acyl halide, R³COCl for the parafluorobenzoyl chloride. Further, Ar′ or Ar′-L may be substituted for 4-aminopyridine in the last step.

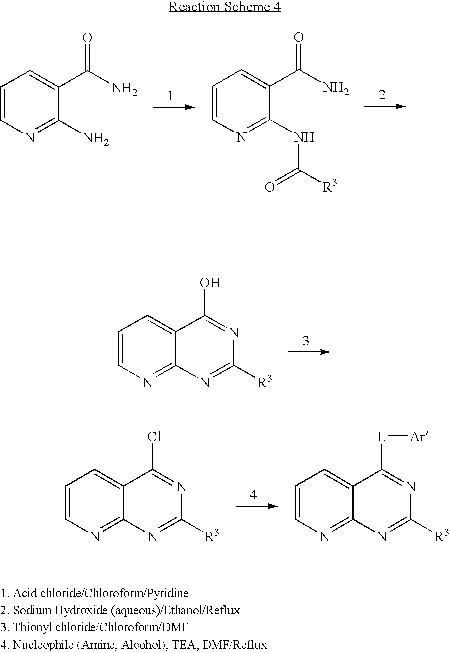

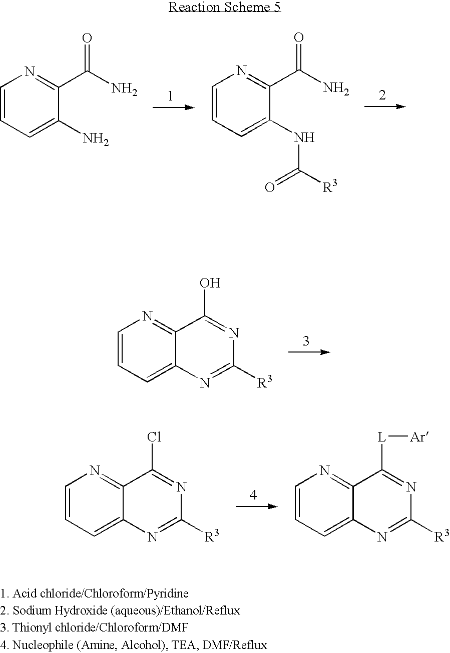

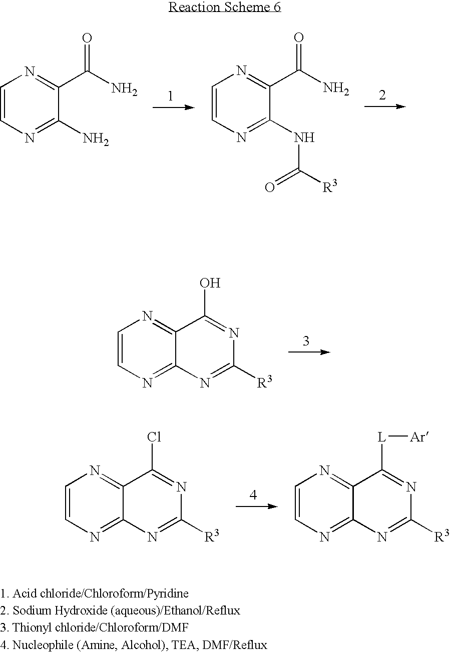

It is seen that Reaction Scheme I represents the last step of Reaction Schemes 2-6 and that Reaction Scheme 2 represents the last two steps of Reaction Scheme 3-6.

Reaction Scheme 7 provides conditions wherein compounds of formula (1) are obtained wherein R¹ is other than H.

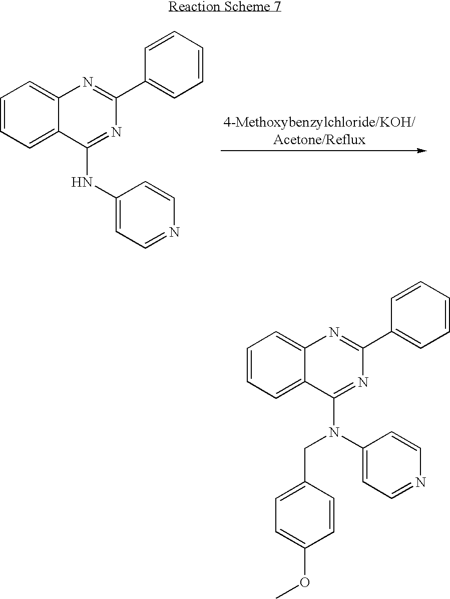

Reaction Scheme 8 is a modification of Reaction Scheme 3 which simply demonstrates that substituents on ring A are carried through the synthesis process. The principles of the behavior of the substituents apply as well to Reactions Schemes 4-6.

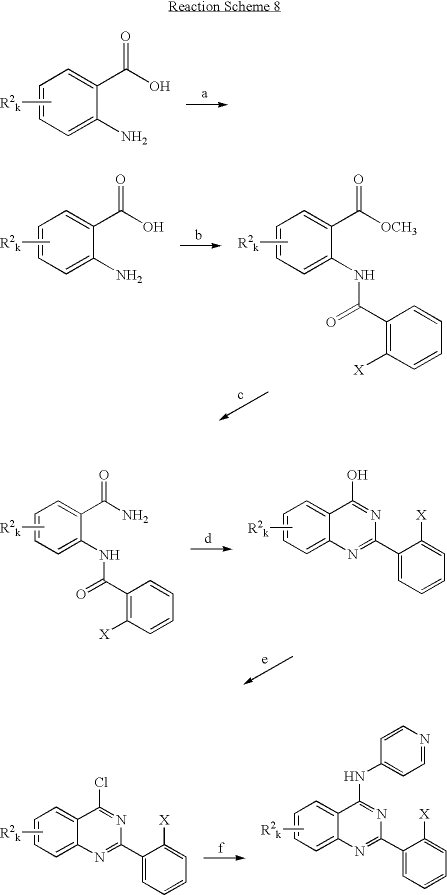

Reaction Scheme 8 shows a modified form of Reaction Scheme 3 which includes substituents R² in the quinazoline ring of formula (1). The substituents are carried throughout the reaction scheme. In step a, the starting material is treated with thionyl chloride in the presence of methanol and refluxed for 12 hours. In step b, the appropriate substituted benzoyl chloride is reacted with the product of step a by treating with the appropriately substituted benzoyl chloride in pyridine for 24 hours. In embodiments wherein X (shown illustratively in the ortho-position) is fluoro, 2-fluorobenzoyl chloride is used as a reagent; where X is (for illustration ortho-chloro), 2-chlorobenzoyl chloride is used.

In step c, the ester is converted to the amide by treating in ammonium hydroxide in an aprotic solvent such as dimethyl formamide (DMF) for 24 hours. The product is then cyclized in step d by treatment with 10 N NaOH in ethanol and refluxed for 3 hours.

The resulting cyclized form is then converted to the chloride in step e by treating with thionyl chloride in chloroform in the presence of a catalytic amount of DMF under reflux for 4 hours. Finally, the illustrated 4-pyridylamino compound is obtained in step f by treating with 4-amino pyridine in the presence of potassium carbonate and DMF and refluxed for 2 hours.

In illustrative embodiments of Reaction Scheme 8, R² may, for example, provide two methoxy substituents so that the starting material is 2-amino-4,5-dimethoxy benzoic acid and the product is, for example, 2-(2-chlorophenyl)-4-(4-pyridylamino)-6,7-dimethoxyquinazoline.

In another illustrative embodiment, R² provides a single nitro; the starting material is thus, for example, 2-amino-5-nitrobenzoic acid and the resulting compound is, for example. 2(2-fluorophenyl)-4-(4-pyridylamino)-5-nitroquinazoline.

Reaction Schemes 4-6 can be carried out in a manner similar to that set forth in Reaction Scheme 8, thus carrying along R² substituents through the steps of the process.

In compounds of the invention wherein R² is nitro, the nitro group may be reduced to amino and further derivatized as indicated in Reaction Scheme 9.

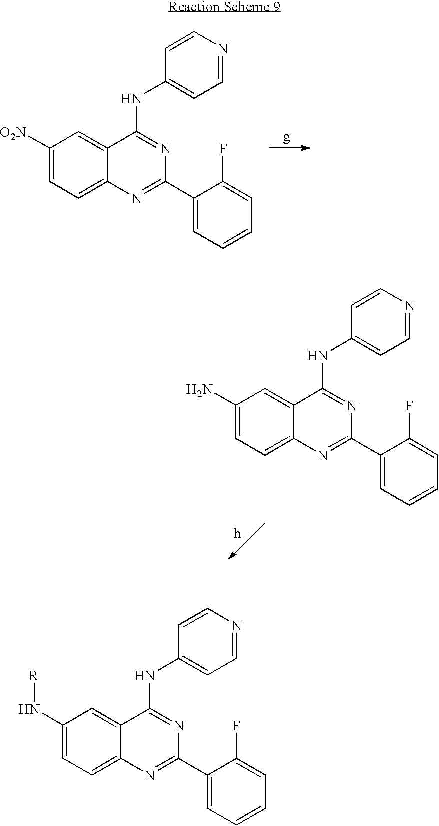

In Reaction Scheme 9, the illustrative product of Reaction Scheme 8 is first reduced in step g by treating with hydrogen and palladium on carbon (10%) in the presence of acetic acid and methanol at atmospheric pressure for 12 hours to obtain the amino compound. The resulting amino compound is either converted to the acyl form (R=acyl) using the appropriate acid chloride in the presence of chloroform and pyridine for four hours, or is converted to the corresponding alkylated amine (R=alkyl) by treating the amine intermediate with the appropriate aldehyde in the presence of ethanol, acetic acid, and sodium triacetoxyborohydride for 4 hours.

While the foregoing exemplary Reaction Schemes are set forth to illustrate the synthetic methods of the invention, it is understood that the substituents shown on the quinazoline ring of the products are generically of the formula (1) as described herein and that the reactants may be substituted accordingly. Variations to accommodate various substituents which represent embodiments of R³ other than the moieties shown in these illustrative examples or as Ar′ in these illustrative examples may also be used. Similarly, embodiments wherein the substituent at position 4 contains an arylalkyl can be used in these schemes. Methods to synthesize the compounds of the invention are, in general, known in the art.

A number of synthetic routes may be employed to produce the compounds of formula (2). In general, they may be synthesized using reactions known in the art. One useful method, especially with regard to embodiments which contain nitrile substitutions (which also, of course, can be hydrolyzed to the corresponding carboxylic acids or reduced to the amines) is shown in Reaction Scheme 10, shown below. In Reaction Scheme 1, an intermediate wherein the pyrimidine ring is halogenated is obtained; the halide is then displaced by an aryl amine. In this method, the pyrimidine ring is generated in the synthetic scheme, resulting in the compound formed in reactions labeled a.

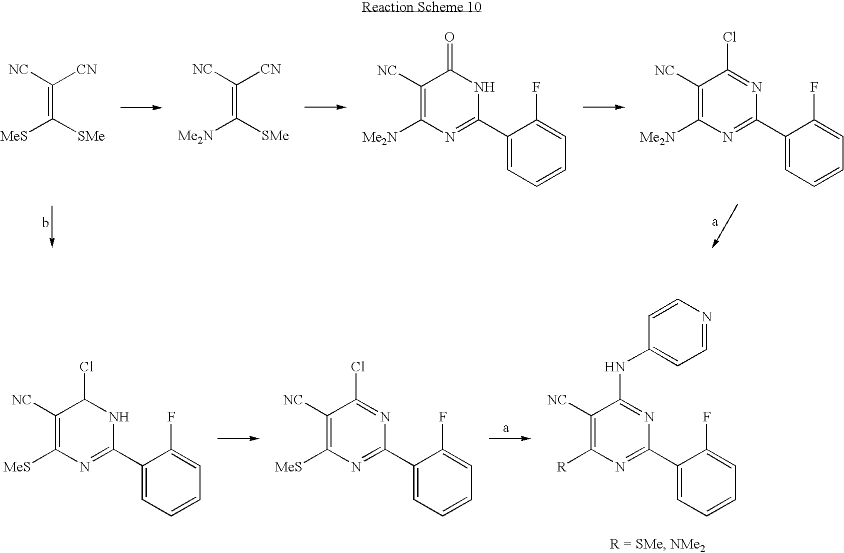

In Reaction Scheme 11, the pyrimidine ring is obtained by cyclizing an amido moiety and, again, a halo group on the pyrimidine ring is displaced by an aryl amide to obtain the compounds of the invention in step b. Further substitution on the resulting invention compound can then also be performed as shown in subsequent steps b¹, b², and b³.

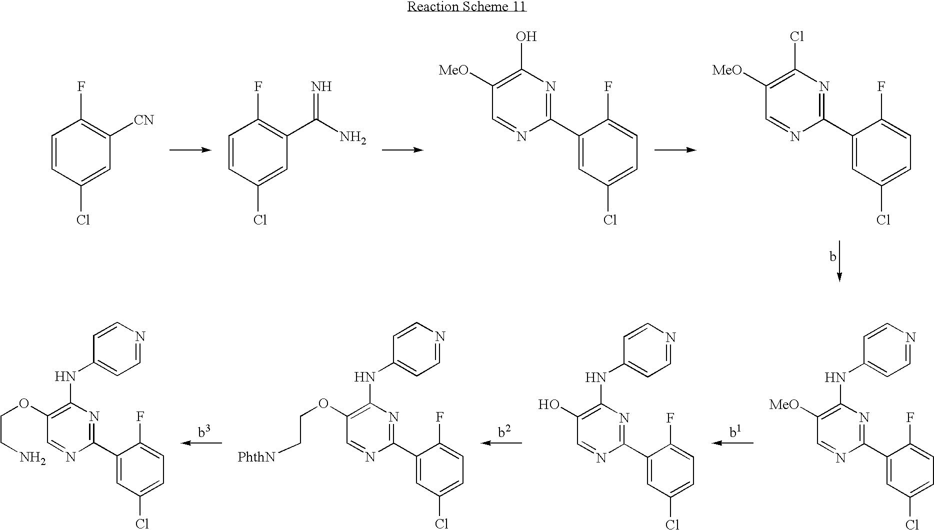

Reaction Schemes 12, 13, 14 and 15, shown below, provide alternative routes to the pyrimidine nucleus, and further substitution thereof.

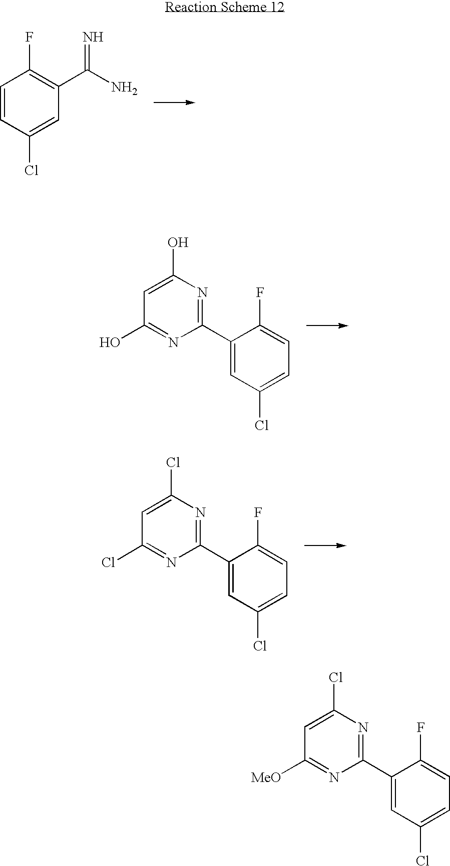

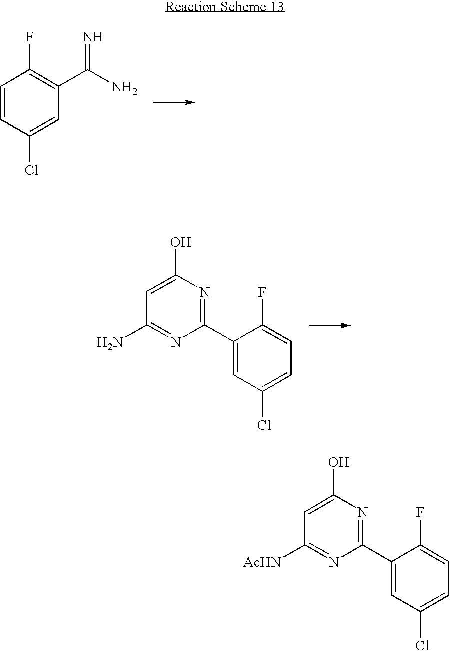

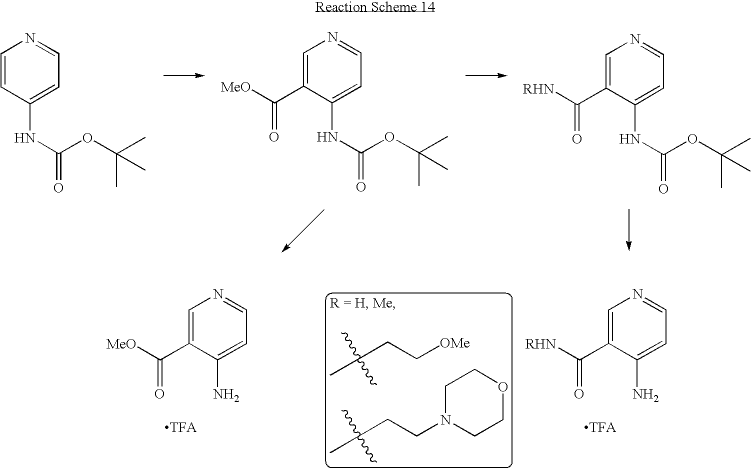

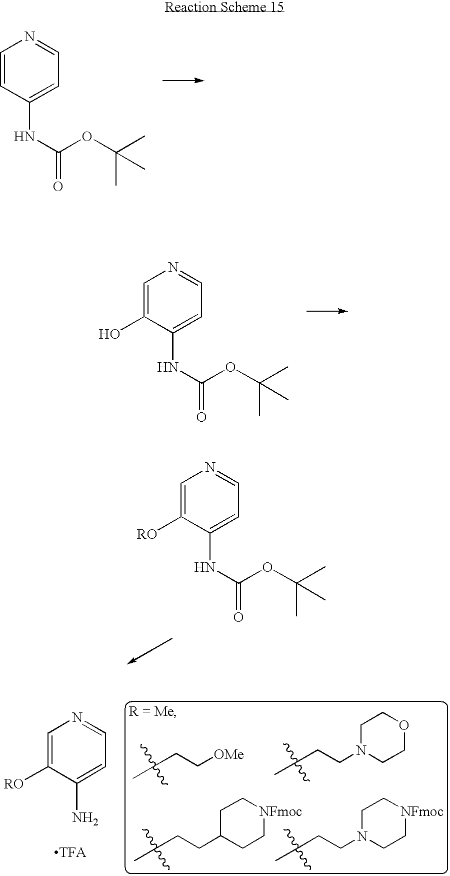

Small organic molecules other than quinazoline derivatives or pyrimidine derivatives can be synthesized by well known methods of organic chemistry as described in standard textbooks.

Activity of the Compounds

Compounds that are useful in the methods of the present invention can be identified by their ability to inhibit TGF-β. An assay for identifying the useful compounds can, for example, be conducted as follows: Compound dilutions and reagents are prepared fresh daily. Compounds are diluted from DMSO stock solutions to 2 times the desired assay concentration, keeping final DMSO concentration in the assay less than or equal to 1%. TGFβ-R1 should be diluted to 4 times the desired assay concentration in buffer+DTT. ATP can be diluted into 4× reaction buffer, and gamma-³³P-ATP can be added at 60μCi/mL.

The assay can be performed, for example, by adding 10 μl of the enzyme to 20 μl of the compound solution. In a possible protocol, the reaction is initiated by the addition of 10 μl of ATP mix. Final assay conditions include 10 uM ATP, 170 nM TGFβ R1, and 1M DTT in 20 mM MOPS, pH 7. The reactions are incubated at room temperature for 20 minutes. The reactions are stopped by transferring 23 μl of reaction mixture onto a phosphocellulose 96-well filter plate, which has been pre-wetted with 15 μl of 0.25M H₃PO₄ per well. After 5 minutes, the wells are washed 4× with 75 mM H₃PO₄ and once with 95% ethanol. The plate is dried, scintillation cocktail is added to each well, and the wells are counted in a Packard TopCount microplate scintillation counter.

Alternatively, compounds can be evaluated by measuring their abilities to inhibit the phosphorylation of the substrate casein. An assay can be conducted as follows: Compound dilutions and reagents are prepared fresh daily. Compounds are diluted from DMSO stock solutions to 2 times the desired assay concentration, keeping final DMSO concentration in the assay less than or equal to 1%. TGF R1 kinase should be diluted to 4 times the desired assay concentration in buffer+DTT. ATP and casein can be diluted into 4× reaction buffer, and gamma-33P-ATP can be added at 50 μCi/mL.

According to a possible protocol, the assay can be performed by adding 10 μl of the enzyme to 20 μl of the compound solution. The reaction is initiated by the addition of 10 μl of the casein/ATP mix. Final assay conditions include 2.5 μM ATP, 100 μM casein, 6.4 nM TGF R1 kinase, and 1M DTT in 20 mM Tris buffer, pH 7.5. The reactions are incubated at room temperature for 45 minutes. The reactions are stopped by transferring 23 μl of reaction mixture onto a phosphocellulose 96-well filter plate, which has been pre-wetted with 15 ul of 0.25M H₃PO₄ per well. After 5 minutes, the wells are washed 4× with 75 mM H₃PO₄ and once with 95% ethanol. The plate is dried, scintillation cocktail is added to each well, and the wells are counted in a Packard TopCount microplate scintillation counter. The ability of a compound to inhibit the enzyme is determined by comparing the counts obtained in the presence of the compound to those of the positive control (in the absence of compound) and the negative control (in the absence of enzyme).

Methods of Treatment

Fibroproliferative diseases that can be treated in accordance with the present invention include, without limitation, kidney disorders associated with unregulated TGF-β activity and excessive fibrosis and/or sclerosis, such as glomerulonephritis (GN) of all etiologies, e.g., mesangial proliferative GN, immune GN, and crescentic GN; diabetic nephropathy; renal interstitial fibrosis and all causes of renal interstitial fibrosis, including hypertension; renal fibrosis resulting from complications of drug exposure, including cyclosporin treatment of transplant recipients, e.g. cyclosporin treatment; HIV-associated nephropathy, transplant necropathy. The invention further includes the treatment of hepatic diseases associated with excessive scarring and progressive sclerosis, including cirrhosis due to all etiologies, disorders of the biliary tree, and hepatic dysfunction attributable to infections such as infection with hepatitis virus or parasites; pulmonary fibrosis and symptoms associates with pulmonary fibrosis with consequential loss of gas exchange or ability to efficiently move air into and out of the lungs, including adult respiratory distress syndrome (ARDS), chronic obstructive pulmonary disease (COPD); idiopathic pulmonary fibrosis (IPF), acute lung injury (ALI), or pulmonary fibrosis due to infectious or toxic agents such as smoke, chemicals, allergens, or autoimmune diseases, such as systemic lupus erythematosus and scleroderma, chemical contact, or allergies. Fibroproliferative diseases targeted by the treatment methods herein further include cardiovascular diseases, such as congestive heart failure, dilated cardiomyopathy, myocarditis, or vascular stenosis associated with atherosclerosis, angioplasty treatment, or surgical incisions or mechanical trauma. The invention also includes the treatment of all collagen vascular disorders of a chronic or persistent nature including progressive systemic sclerosis, polymyositis, scleroderma, dermatomyositis, fascists, or Raynaud's syndrome, or arthritic conditions such as rheumatoid arthritis; eye diseases associated with fibroproliferative states, including proliferative vitreoretinopathy of any etiology or fibrosis associated with ocular surgery such as treatment of glaucoma, retinal reattachment, cataract extraction, or drainage procedures of any kind; excessive or hypertrophic scar formation in the dermis occurring during wound healing resulting from trauma or surgical wounds.

The manner of administration and formulation of the compounds useful in the invention and their related compounds will depend on the nature of the condition, the severity of the condition, the particular subject to be treated, and the judgement of the practitioner; formulation will depend on mode of administration. The small molecule compounds of the invention are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of about 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis or ophthalmic treatments, or in formulation intended to penetrate the skin or eye. These include lotions, creams, ointments, drops and the like which can be formulated by known methods.

The compounds may also be administered by injection, including intravenous, intramuscular, subcutaneous, intrarticular or intraperitoneal injection. Typical formulations for such use are liquid formulations in isotonic vehicles such as Hank's solution or Ringer's solution.

Alternative formulations include aerosol inhalants, nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100mg/kg total body weight, preferably from 0.01-50 mg/kg and more preferably about 0.01 mg/kg-10 mg/kg. The dose regimen will vary, however, depending on the conditions being treated and the judgment of the practitioner.

It should be noted that the compounds useful for the invention can be administered as individual active ingredients, or as mixtures of several different compounds. In addition, the TGF-β inhibitors can be used as single therapeutic agents or in combination with other therapeutic agents. Drugs that could be usefully combined with these compounds include natural or synthetic corticosteroids, particularly prednisone and its derivatives, monoclonal antibodies targeting cells of the immune system or genes associated with the development or progression of fibrotic diseases, and small molecule inhibitors of cell division, protein synthesis, or mRNA transcription or translation, or inhibitors of immune cell differentiation or activation.

As implicated above, although the compounds of the invention may be used in humans, they are also available for veterinary use in treating non-human mammalian subjects.

Further details of the invention will be apparent from the following non-limiting examples.

### EXAMPLE 1

Blocking Profibrotic Responses of Primary Human and Rat Lung Fibroblasts to TGF-β Pathway Activation by TGFβ-R1 Kinase Inhibitors

As discussed before, TGF-β plays a central role in wound healing a fibrosis. Lung fibroblasts are key mediators of fibrosis in pulmonary models such as bleomycin-treated rats and in human diseases such as scleroderma, idiopathic pulmonary fibrosis, and chronic obstructive pulmonary disease. Inhibition of TGF-β signaling presents a novel treatment paradigm for pathological fibrotic processes.

The effects of TGFβ-R1 kinase inhibitors on profibrotic gene and protein expression by fibroblasts isolated from human or rat lung was studied.

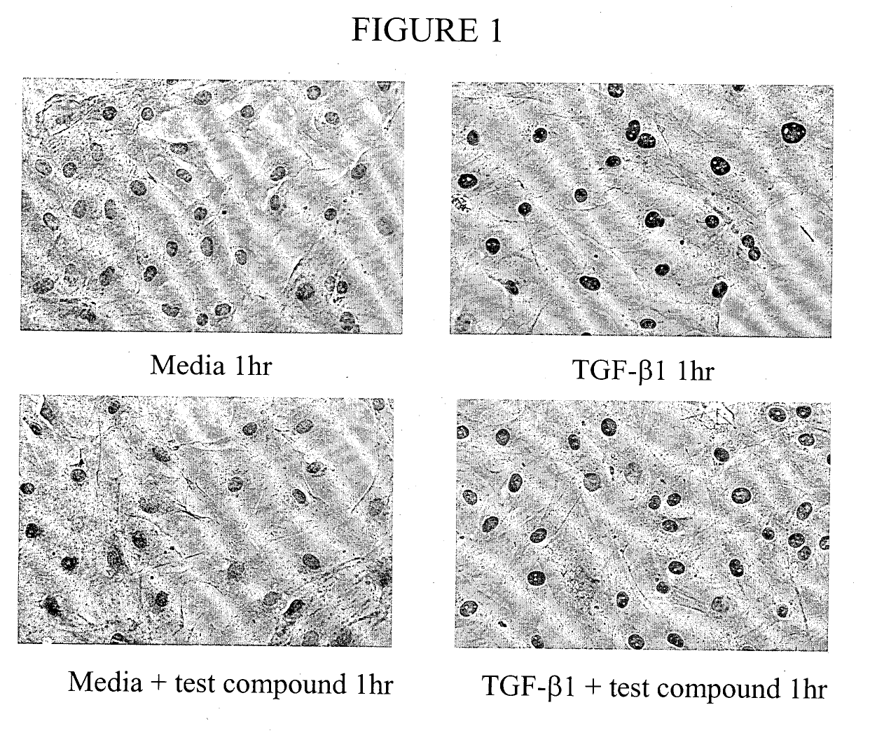

In order to study the effect of TGF-β inhibitors selectively binding to the TGFβ-R1 kinase receptor on TGF-β-induced translocation of Smad2/3 to the nucleus, isolated rat lung fibroblast cells (RLF) were serum starved for 24 hours, then treated with 15 ng/ml TGF-β±0.1 μM of an inhibitor provided in the Tables above, fixed and stained with anti-Smad2/3 monoclonal antibody, and developed using the ABC method which employs biotinylated antibody and a preformed Avidin: Biotinylated enzyme Complex (hence the name“ABC”). The inhibitors block TGF-β induced Smad2/3 translocation to the nucleus in RLF. Illustrative immunohistochemistry results are shown in FIG. 1. Other compounds listed in Tables 1-5 have similar effects.

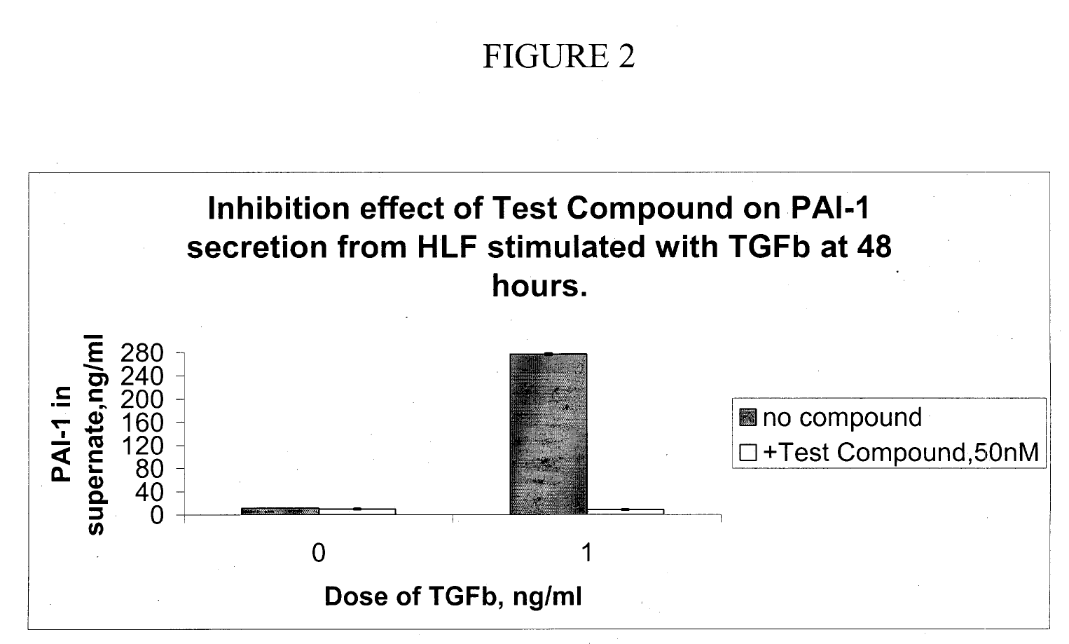

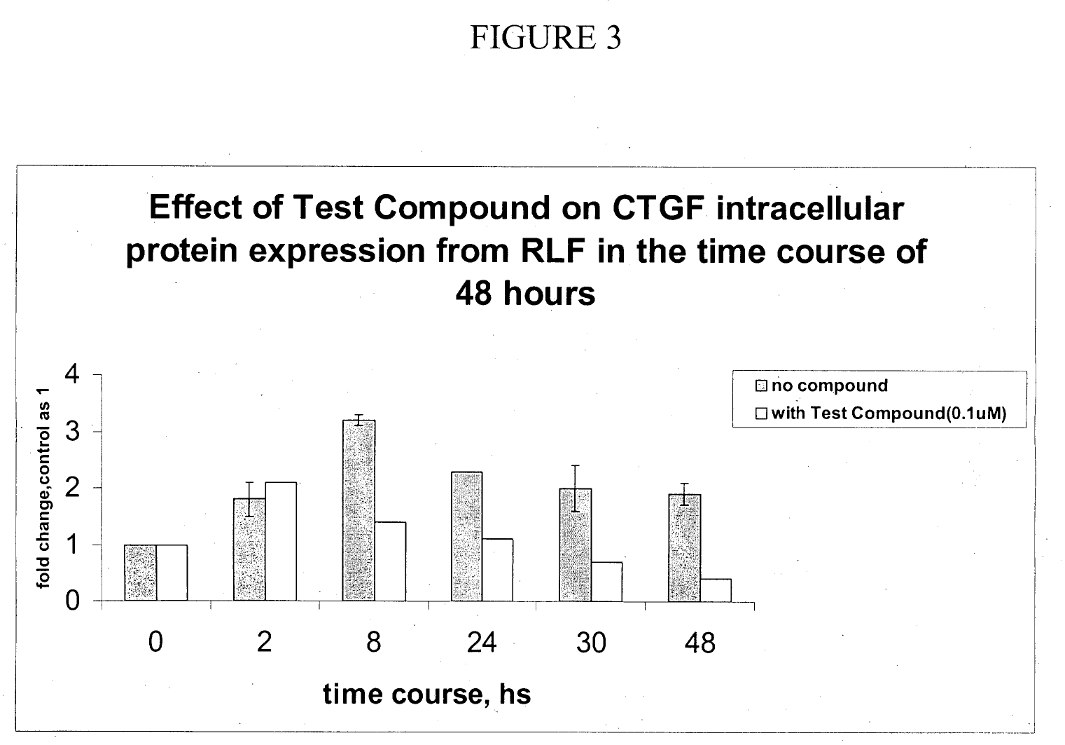

In separate experiments, the effect of the inhibitors, such as those listed in Tables 1-5, on the expression levels of certain profibrotic proteins in RLF was tested, as measured by RT-PCR described in Example 2 below. Treatment with TGF-β resulted in elevation of the expression levels of profibrotic proteins plasminogen activator inhibitor-1 (PAI-1), and connective tissue growth factor (CTGF) as measured by RT-PCR (Taqman). Although bleomycin induced CTGF in culture, reversal with inhibitors had not been demonstrated. All of these effects were reversed by treatment with about 0.1-1 μM of the TGF-β inhibitors. In similar studies using fibroblasts isolated from human lung (HLF), TGF-β induced PAI-1 protein secretion, which could be inhibited by the TGF-β inhibitors. Illustrative results are shown in FIGS. 2 and 3.

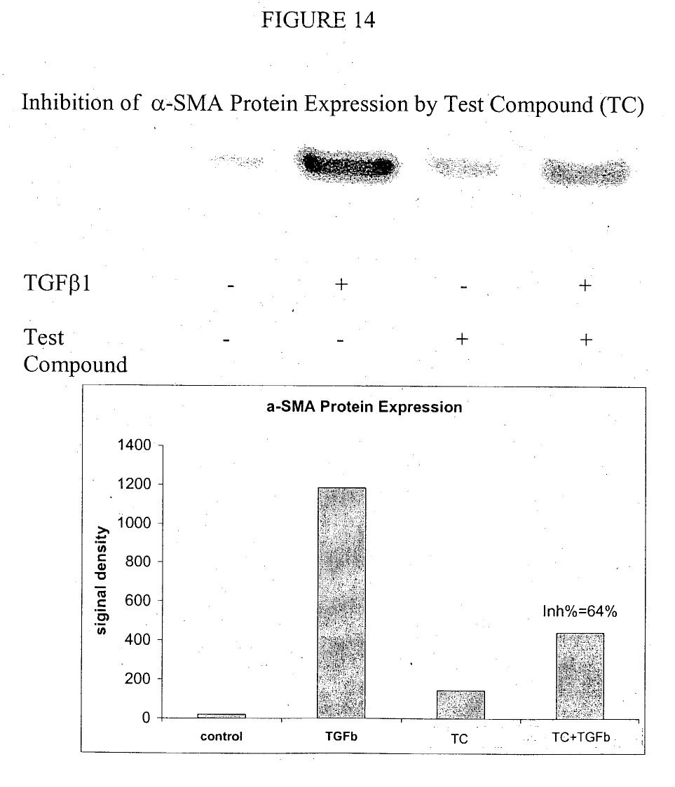

In a similar experiment, human lung fibroblasts (HLF) were stimulated by 3 mg/ml of TGF-β for 3 days, and co-treated with 400 nM of a representative test compound selected from compounds listed in Tables 1-5 above. α-SMA protein expression was measured by Western Blotting. As shown in FIG. 14, treatment with the test compound significantly inhibited α-SMA protein expression in this assay.

In another experiment, the effects of a representative test compound selected from compounds listed in Tables 1-5 above on the regulation of glucocorticoid receptor and genes regulated by TGF-β in HLF were studied.

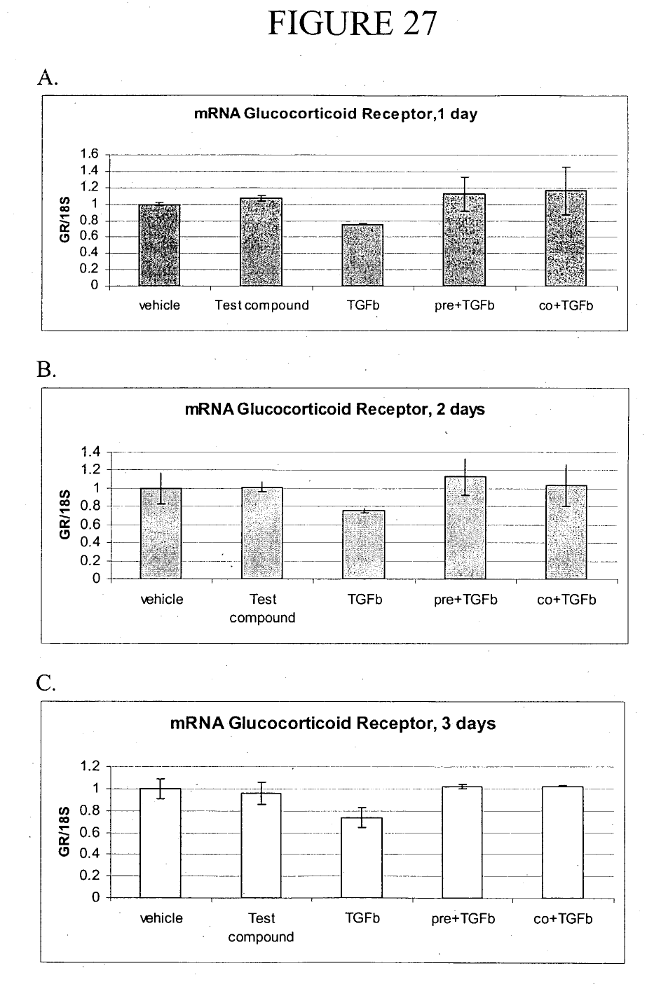

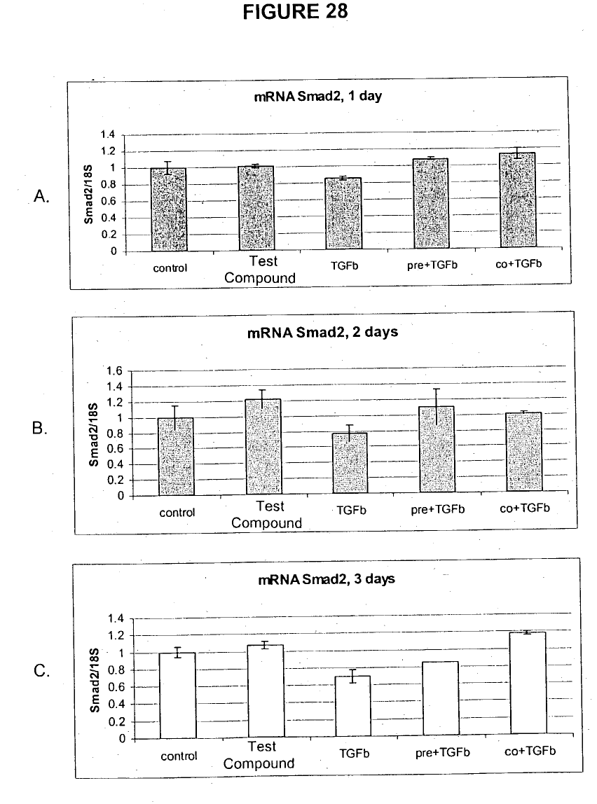

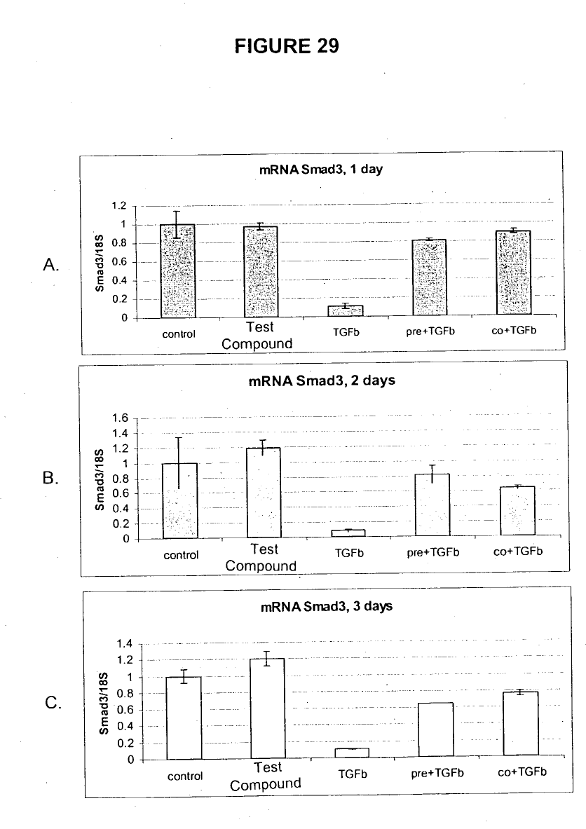

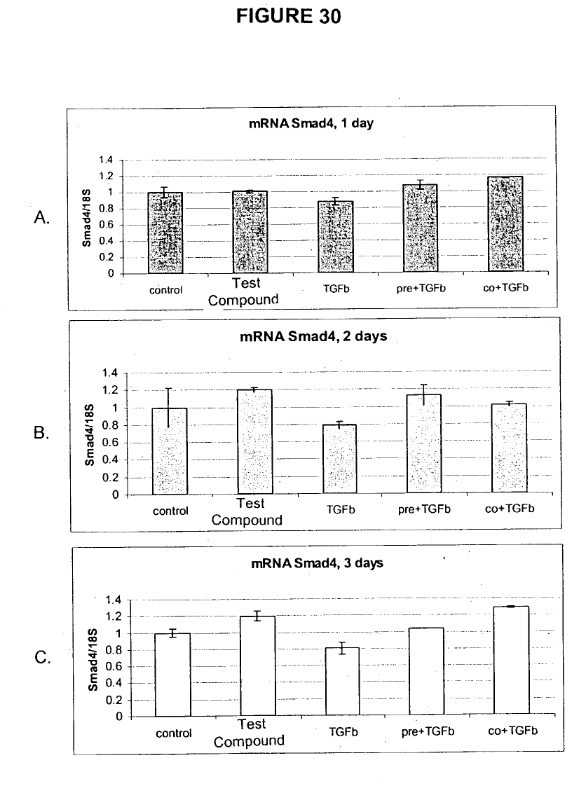

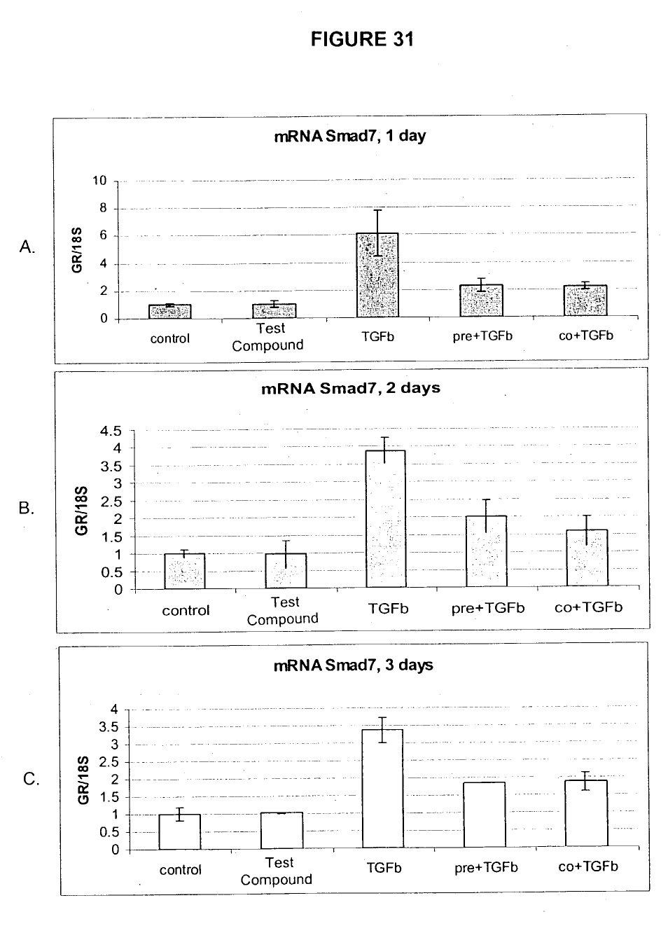

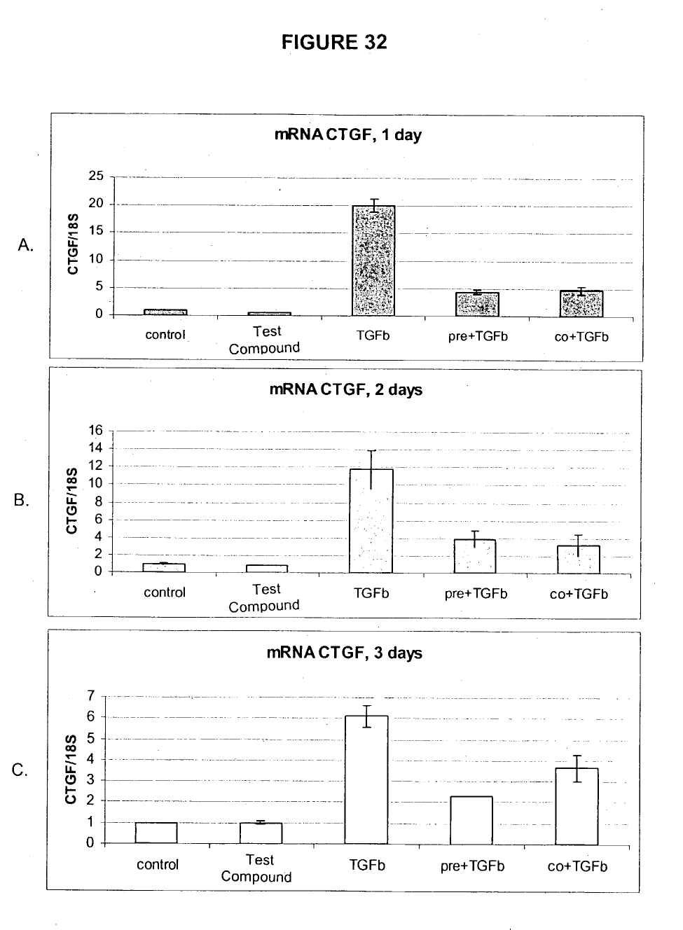

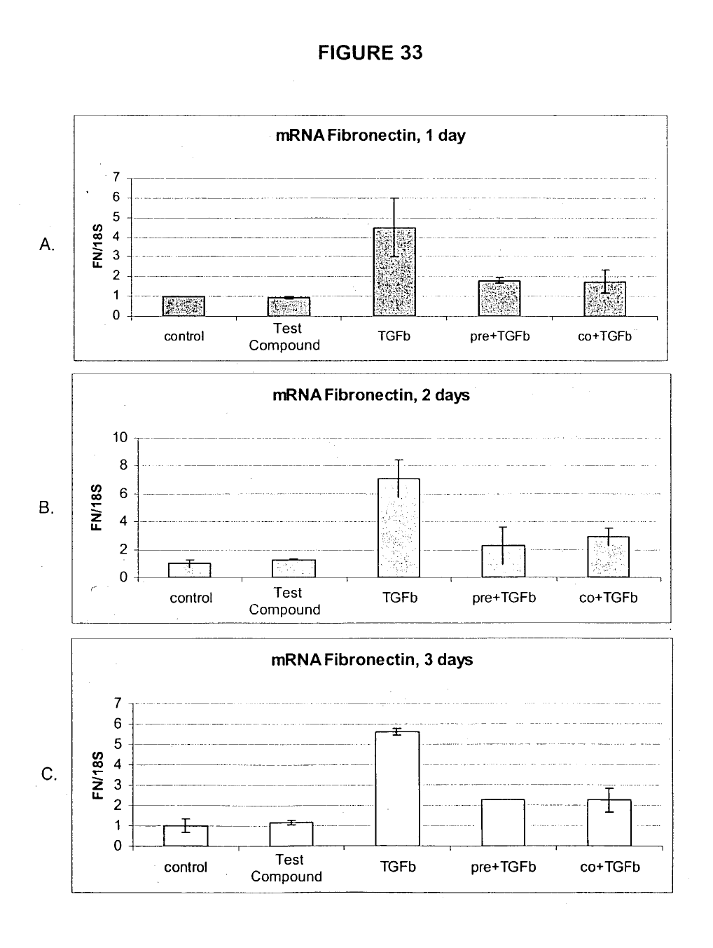

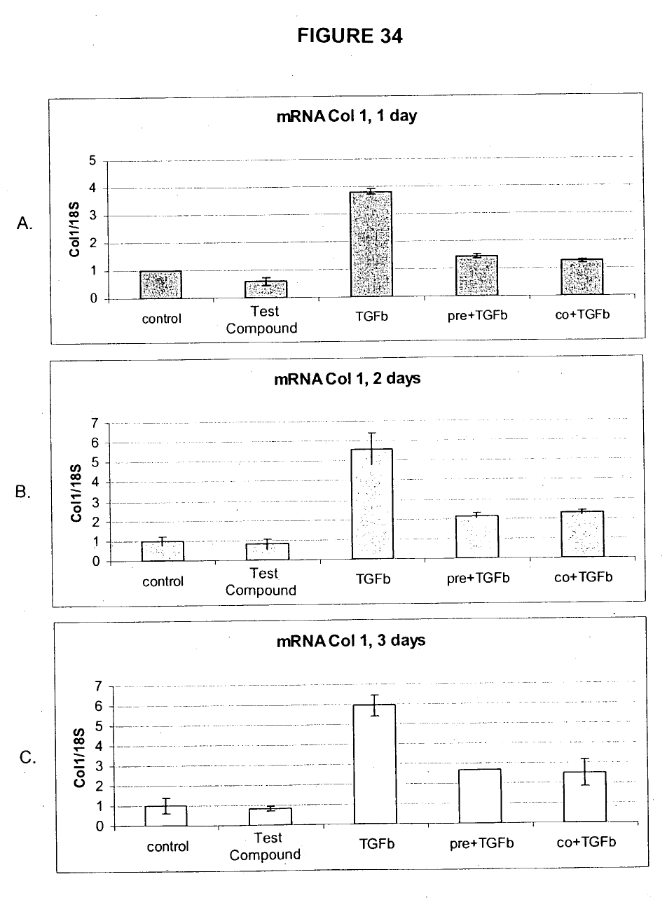

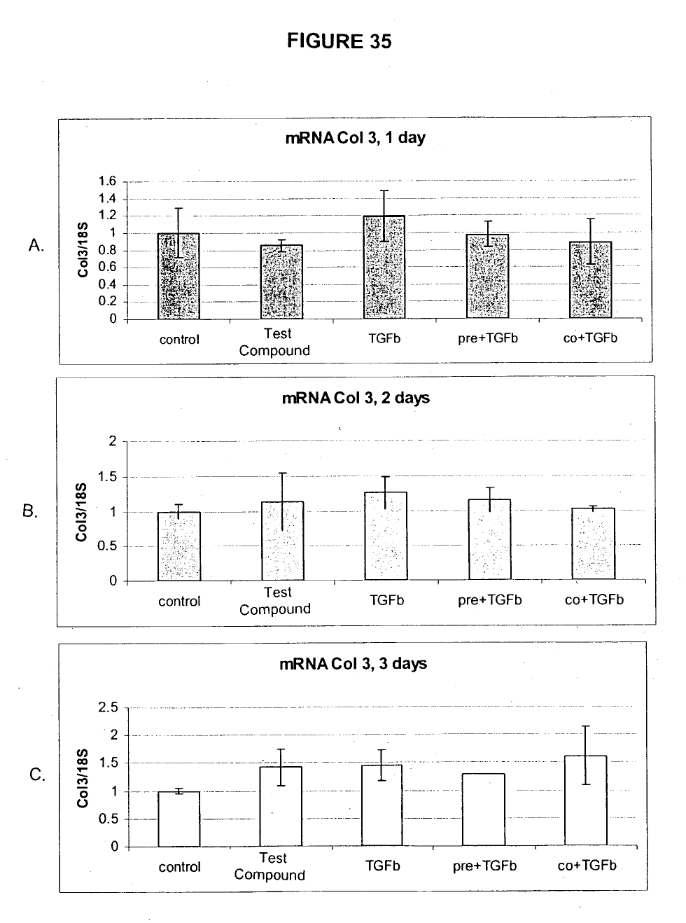

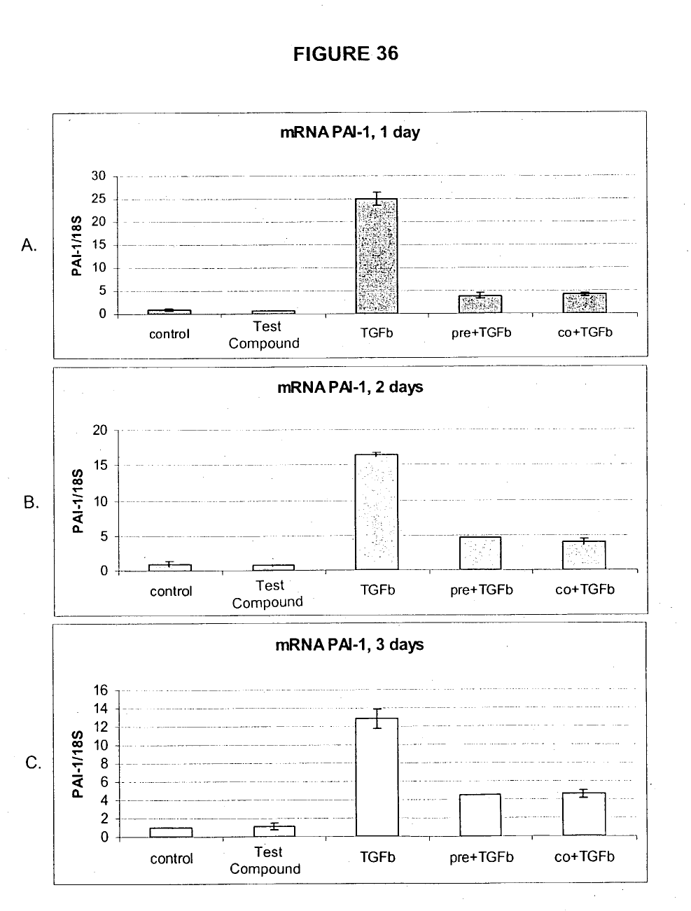

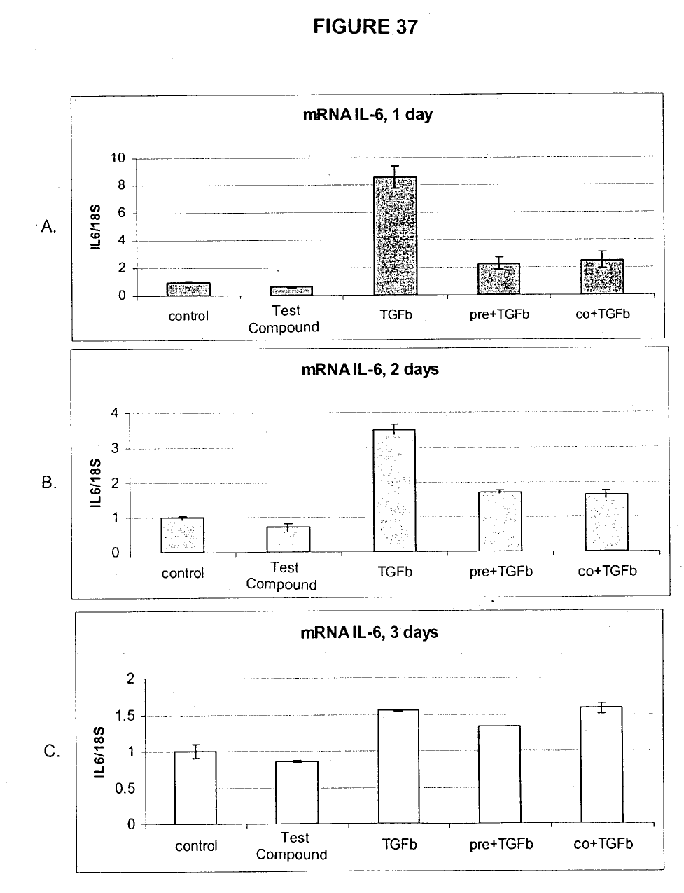

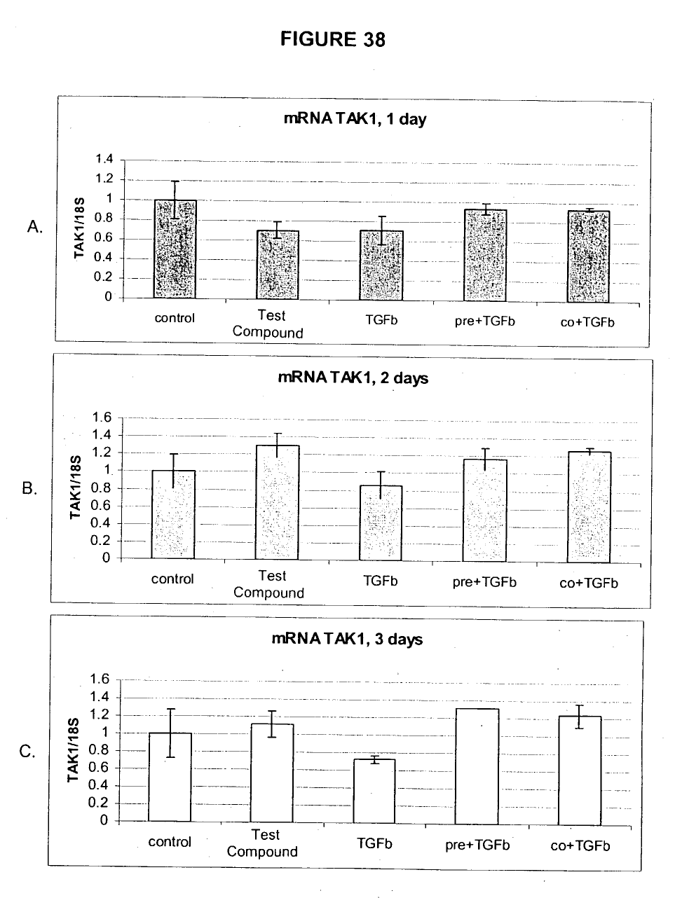

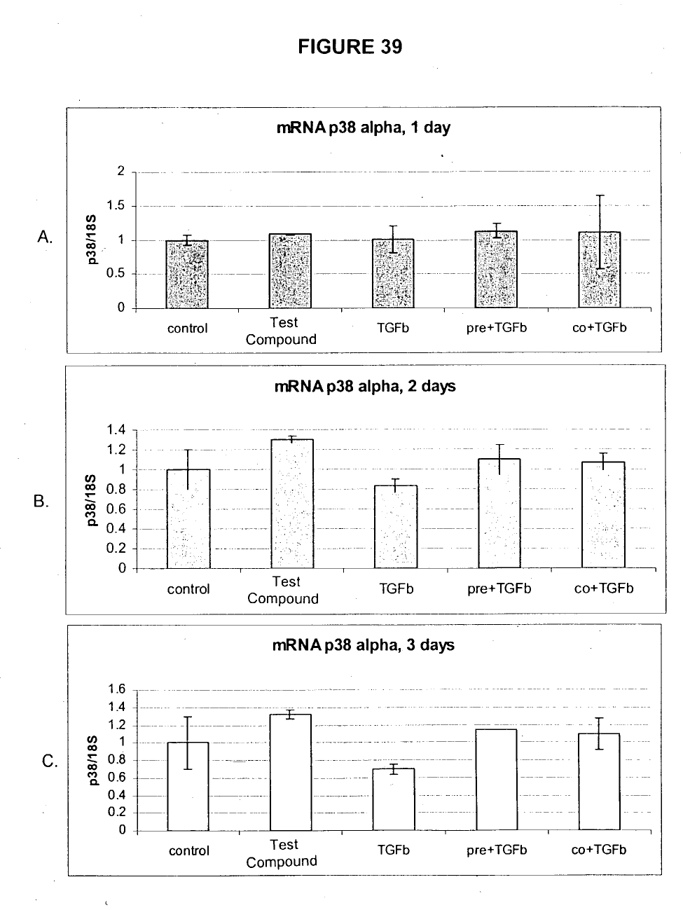

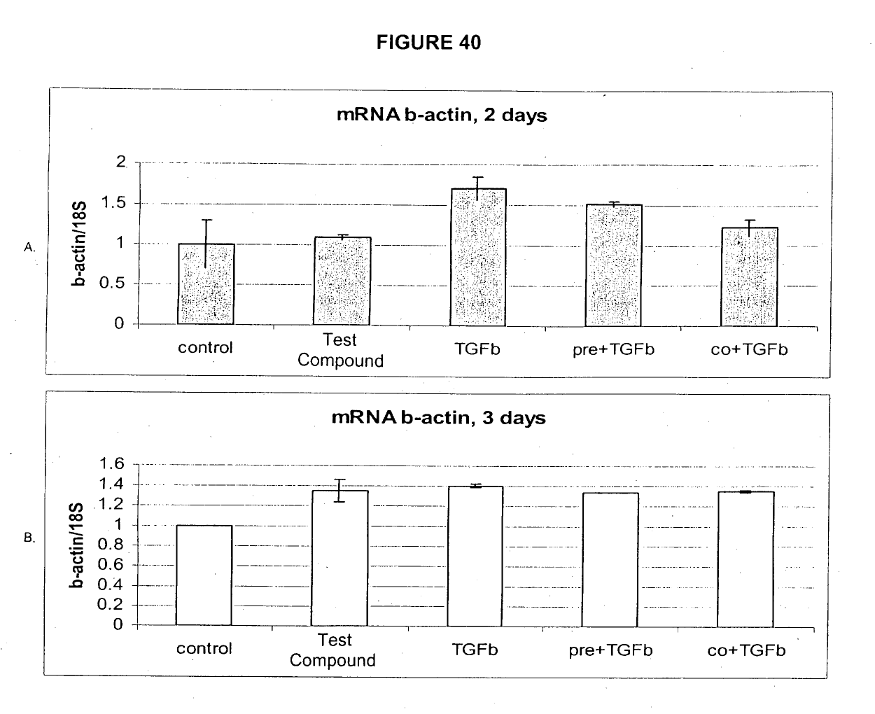

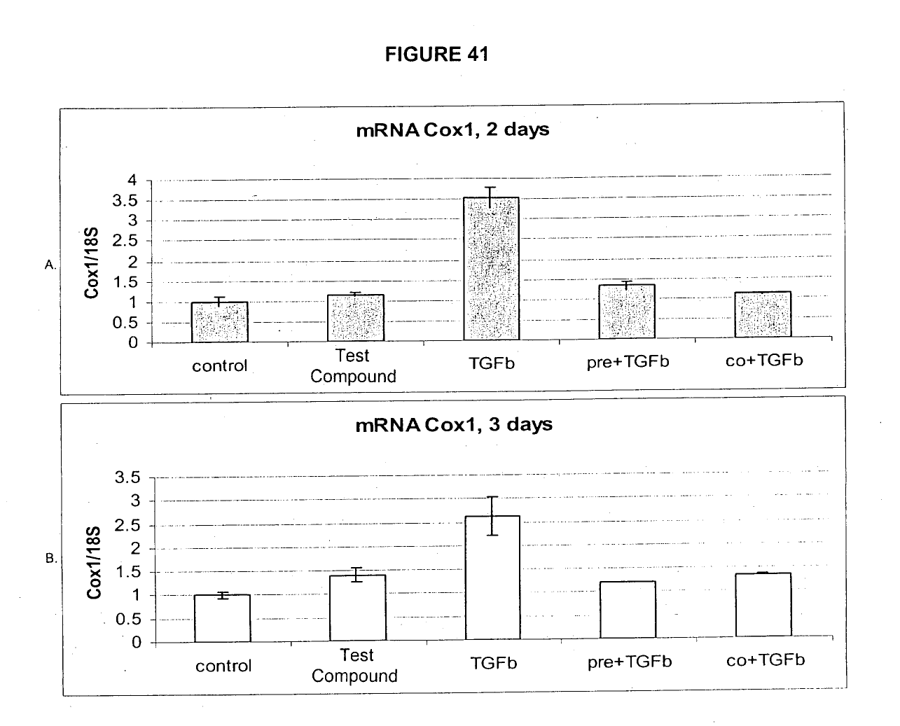

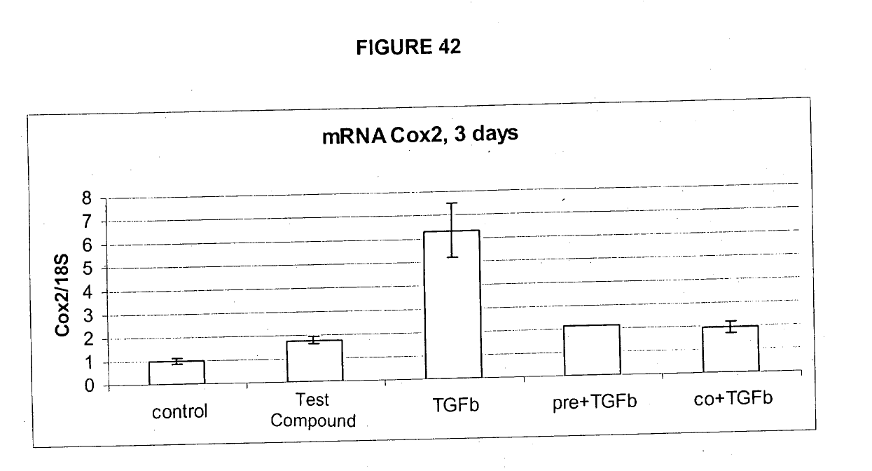

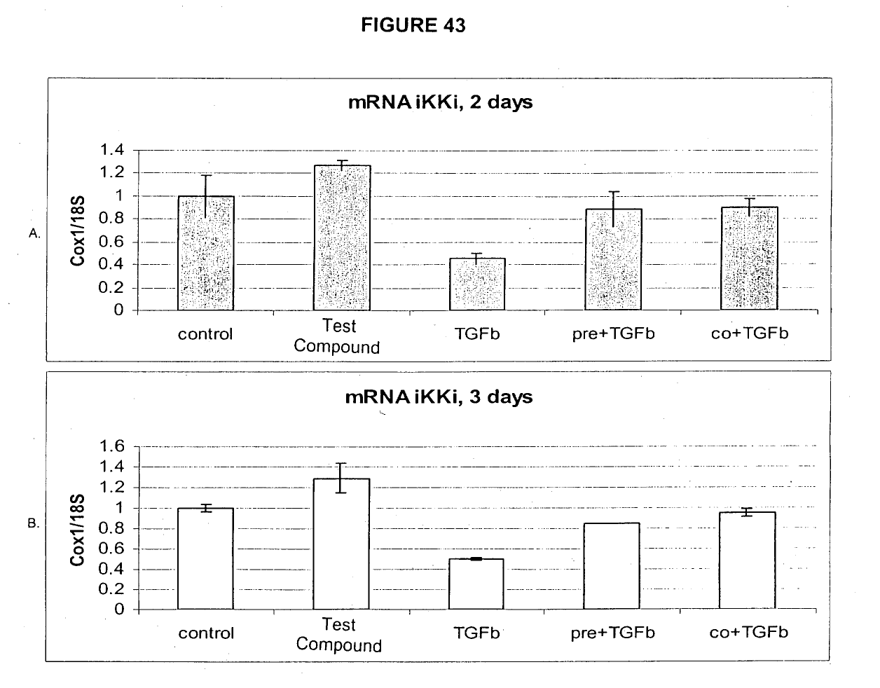

In order to study the effects of the representative test compound on the regulation of glucocorticoid receptor and genes regulated by TGF-β in HLF 7191-94, HLF-40F cells were plated in a 6-well plate, 10⁵ cells/well, 10 wells per time point. The isolated HLF were then serum starved for 24 hours, and the cells were (1) pretreated with 400 nM of a representative test compound or DMSO for 20 minutes, and 5 ng/ml of TGF-β or D′PBS were added, and (2) co-treated with 400 nM of a representative test compound for 1-3 days. The supernatants and cells were collected after each day of the 1-3 day pretreatment and co-treatment, RNA were extracted, and the expression level of the interested mRNA was measured by RT-PCT (Taqman). The mRNA expression levels of glucocorticoid receptor and the genes regulated by TGF-β in HLF at various time points are shown in FIGS. 27-43.

The experiments show that in HLF, at mRNA level, down regulation of glucocorticoid receptor (GR), Smad3 and inducible I kappa-B kinase (iKKi) by TGF-β was inhibited by the test compound. Furthermore, the up-regulation of Smad7, CTGF, fibronectin (FN), Col 1, PAI-1, IL-6, Cox1 and Cox2 was inhibited by the test compound. The mRNA levels of Smad2, Smad4, Col 3, TAK1, p38 alpha (p38a) and α-actin that were treated for 3 days were not significantly affected by TGF-β. There was possible suppression of Smad2, p38a, TGF-β activated kinase 1 (TAK1) by TGF-β after 3 days.

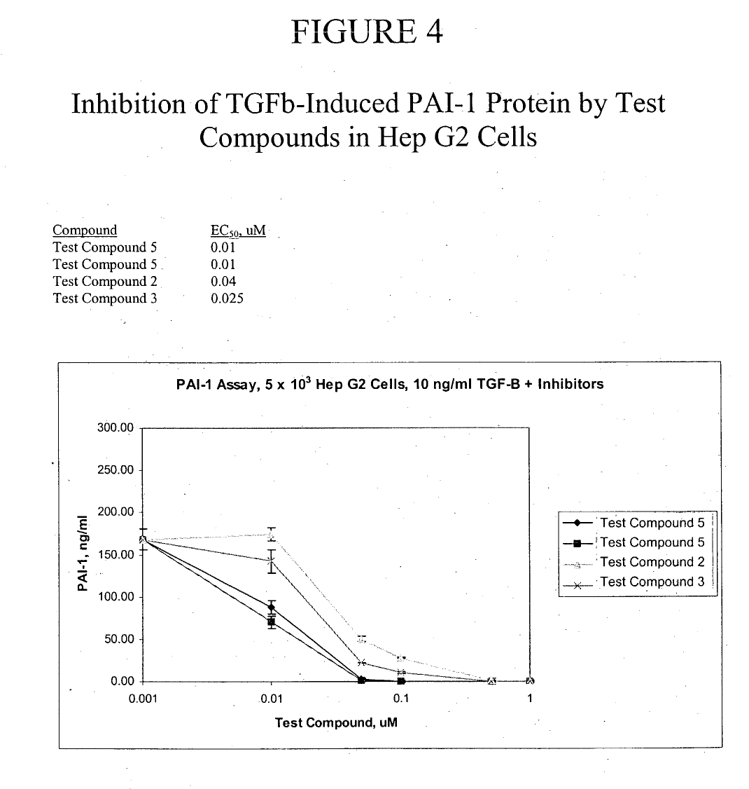

In another experiment, inhibition of TGF-β induced PAI-1 protein expression in 5×10³ HepG2 cells by compounds provided herein. TGF-β was typically employed in a 10 ng/ml concentration, while the amount of the test compounds varied, and typically was in the μM range or below. The compounds inhibited TGF-β induced PAI-1 protein expression. Typical results are shown in FIG. 4.

### EXAMPLE 2

Microarray Gene Expression Profiling

Qantitative Real-Time PCR

Total RNA was analyzed by quantitative real-time PCR (Gibson UEM, Heid C A and Williams P M. A novel method for real time quantitative RT-PCR. Genome Res. 6:995-1001 (1996)) using ABI Prism™ 7700 Sequence Detection System (PE Applied Biosystems Foster City, Calif.). This system is based on the ability of the 5′ nuclease activity of Taq polymerase to cleave a nonextendable dual-labeled fluorogenic hybridization probe during the extension phase of PCR. The probe is labeled with reporter fluorescent dye at the 5′ end and a quencher fluorescent dye (6-carboxy-tetramethyl-rhodamine) at the 3′ end. When the probe is intact, reporter emission is quenched by the physical proximity of the reported and quencher fluorescent dyes. However, during the extension phase of PCR, the nucleolytic activity of the DNA polymerase cleaves the hybridization probe and releases the reporter dye from the probe with a concomitant increase in reporter fluorescence.

The following sequence specific primers and probes were designated using Primer Express software (PE Applied Biosystems, Foster City, Calif.):

| | | | |
|---|---|---|---|
| Gene | Forward primer | Reverse primer | Probe |

| | | | |
|---|---|---|---|
| PAI-1 | 5′-ACTGCACAGGAAGGTAACGTGAA | 5′-GGTTTTCCAGTGGAGATGTAACGGA | 5′-CTAATTTCATAGCGGGCCGCTCTGC |
| | (SEQ ID NO: 1) | (SEQ ID NO: 2) | (SEQ ID NO:3) |
| TIMP-1 | 5′-GGGTCTCGATGACCCGAAG | 5′-AACGGAGGAAAGGTAAACAGTGTGTT | 5′-TTCCCCTGGCAAAAGCTGAACCCT |
| | (SEQ ID NO: 4) | (SEQ ID NO: 5) | (SEQ ID NO: 6) |
| Osteo- | 5′-CCTTCACTGCCAGCACACAA | 5′-GGCCGTCAGGGACATCG | 5′-CGTTTTGACTCCAATCGCCCCCA |
| pontin | (SEQ ID NO: 7) | (SEQ ID NO: 8) | (SEQ ID NO: 9) |
| CTGF | 5′-TCTTCGGTGGGTCCGTGTA | 5′-CCACGGCCCCATCCA | 5′-CGCAGCGGCGAGTCCTTCCA |
| | (SEQ ID NO: 10) | (SEQ ID NO: 11) | (SEQ ID NO: 12) |
| Fibro- | 5′-GCTGCTGGGACTTCCTACGT | 5′-TCTGTTCCGGGAGGTGCA | 5′-TGGGCGAAGGCAATGGGCGTAT |
| nectin | (SEQ ID NO: 13) | (SEQ ID NO: 14) | (SEQ ID NO: 15) |
| 18s | 5′-CGGCTACCACATCCAAGGAA | 5′-GCTGGAATTACCGCGGCT | 5′-TGCTGGCACCAGACTTGCCTC |
| | (SEQ ID NO: 16) | (SEQ ID NO: 17) | (SEQ ID NO: 18) |

Primers were used at a concentration of 200 nM and probes at 100 nM in each reaction. Multiscribe reverse transcriptase and AmpliTaq Gold polymerase (PE Applied Biosystems, Foster City, Calif.) were used in all RT-PCR reactions. RT-PCR parameters were as follows: 48° C. for 30 minutes (reverse transcription), 95° C. for 10 minutes (AmpliTaq Gold activation) and 40 cycles of 95° C. for 15 seconds, 60° C. for 1 minute. Relative quantitation was calculated using the comparative threshold cycle number for each sample fitted to a five point standard curve (ABI Prism 7700 User Bulletin #2, PE Applied Biosystems, Foster City, Calif.). Expression levels were normalized to 18S.

Gene Expression Profiling

Microarrays were fabricated that contained 9,600 cDNA clones isolated from randomly collected from a normalized cDNA libraries or purchased from Research Genetics. Briefly, 9,600 cDNA inserts were generated by PCR amplification with primers derived from flanking vector sequences, purified by gel filtration over Sephacryl 400 (Amersham), dried by lyophilization, and resuspended in 10 μl of 2×SSC. PCR products were arrayed from 96-well microtiter plates onto sialylated microscope slides in an area of 1.8 cm² using print tips constructed as elongate capillary channels, and driven by high-speed robotics. Fluorescently labeled probe pairs were applied to the microarray and allowed to hybridize to each of the 9,600 elements. Degree of hybridization at each element was quantified by sequential excitation of the 2 fluorophores with a scanning laser read at an appropriate wavelength for each emission. Differential expression values were expressed as a ratio of intensities from the two emissions where positive and negative values indicated an increase or decrease, respectively, relative to control. Expression data for particular target genes were rejected if neither channel produced a signal at least 2.5-fold over local background or if the signal derived from less than 40% of the area of the printed spot.

Normal Rat Kidney Cells

Normal rat kidney cells (NRK) were cultured in DMEM-21 (high glucose)10% FCS at 37° C., 5% CO2. Cells were serum starved for 24 hours, before treated with 5 ng/ml huTGF-β1 (R&D System) ± inhibitors for additional 24 hours. Media were removed and cells were washed with PBS for total RNA extraction.

Results

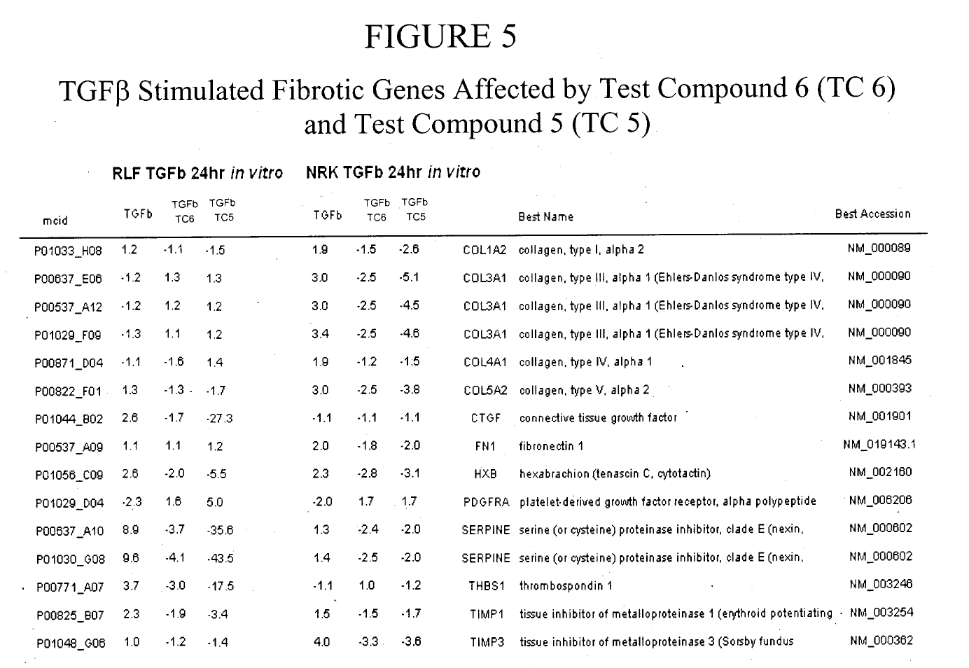

FIG. 5 shows the results of a representative microarray gene expression profiling analysis of cultured rat lung fibroblasts (RLF) and normal rat kidney cells (NRK). Expression of the listed fibrotic genes was altered by TGF-β treatment at 24 hours and was reversed by co-treatment with certain TGF-β inhibitors selected from the compounds listed in the Tables above.

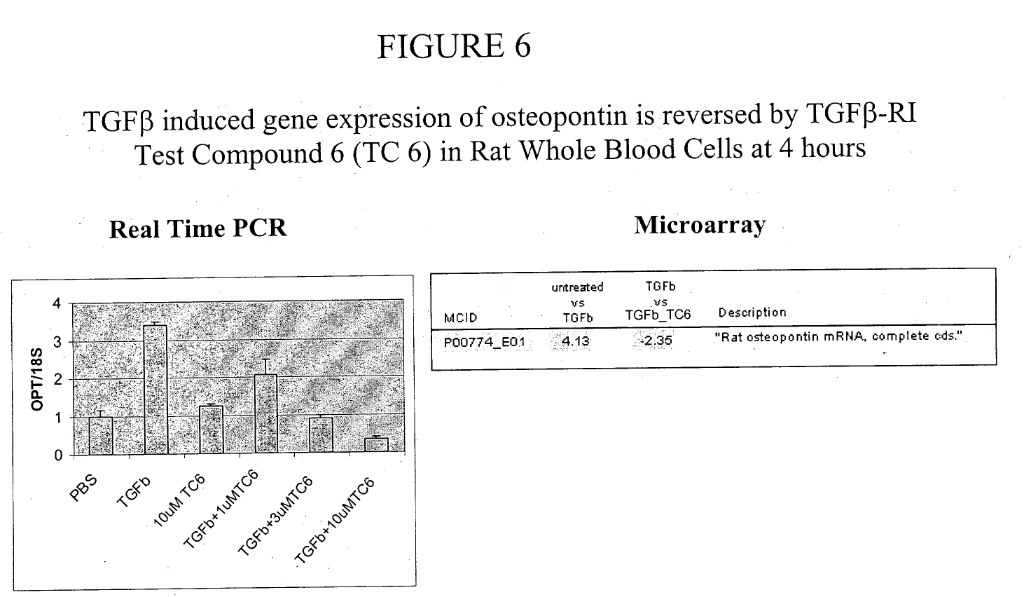

FIG. 6 shows the results of a representative microarray gene expression profiling analysis and quantitative real time PCR of rat whole blood cells. TGF-β induces osteopontin gene expression at 4 hours and this induction is blocked by a representative of the TGFβ-R1 inhibitors provided in the Tables above.

### EXAMPLE 3

Effect of TGF-β Inhibitors on Profibrotic Gene Expression in the Bleomycin Rat Model of Pulmonary Fibrosis

Material and Methods:

Animal information: 275-325 grams, male, Sprague-Dawley rats. Rats were anesthetized with 1.3 ml/kg of 0.8 mg/ml ketamine and 0.5 mg/ml xylazine cocktail. Once the rats were anesthetized, a 16G×2″ Surflo I.V. catheter was inserted into the trachea, 0.5 ml of saline or 0.5 ml of 1.0 units/ml of bleomycin was slowly delivered into the lungs via a 8.5 cm polyethylene tubing PE-50 attached to a 23G×1″ needle which was connected to a 1.0 ml syringe. After the intratracheal administration of saline or bleomycin, two rats were housed in a new cage with new bedding and free access to food and water. Twenty four hours after the incubation, rats were weighted and orally dosed with 5 ml/kg of 1% methyl cellulose (MC) or 5 ml/kg of 2.0, or 6.0 mg/ml test compound twice a day for 4 and a half days or intraperitoneal injection of 2 ml/kg of 4.0 mg/ml of dexamethasone every other day for four and a half day. On day 1, 3, and 5, 400 μl of blood were collected from each rats via the tail to determine the circulation level of test compound. On day 5, rats were sacrificed and bronchoalveolar lavage fluids (BALF) were collected for protein (BCA Protein Assay Kit from Pierce (Cat #: 23225)) and interleukin 6 (R&D System Quantikine® M Rat IL-6 Immunoassay (Cat #: R6000)) analysis, and lung tissues were also collected for Taqman analysis. The test compound in these experiments is selected from Tables 1-5.

Results

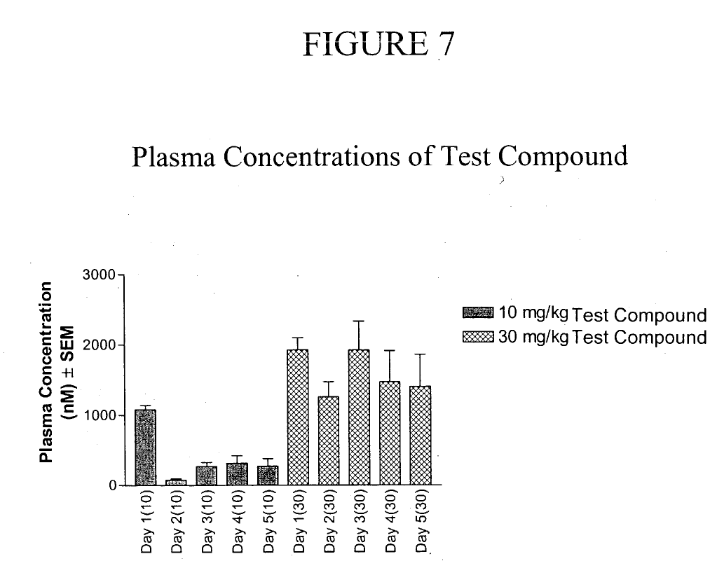

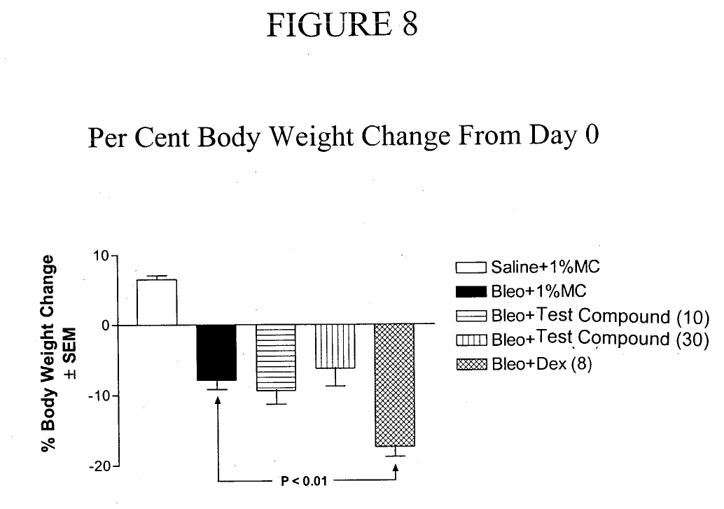

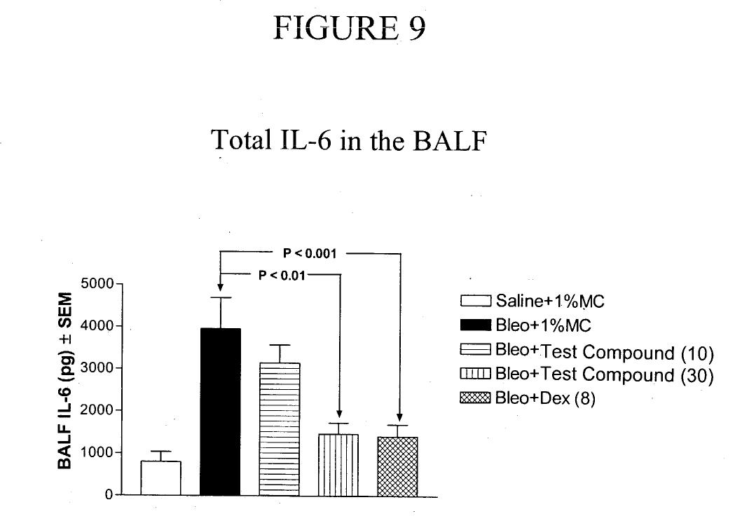

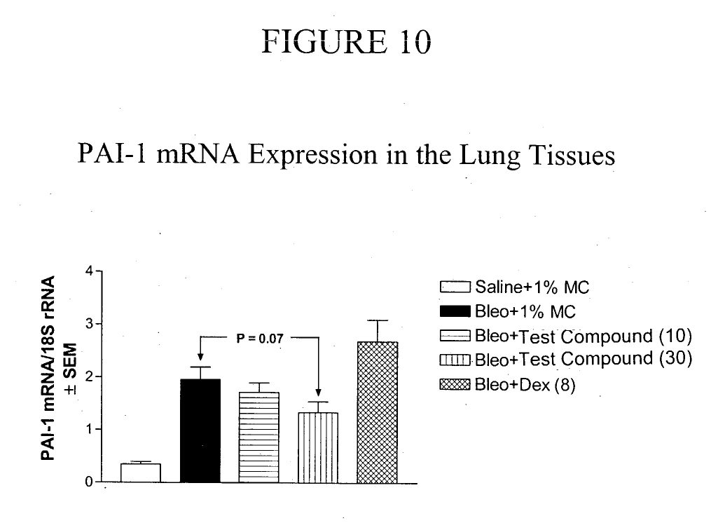

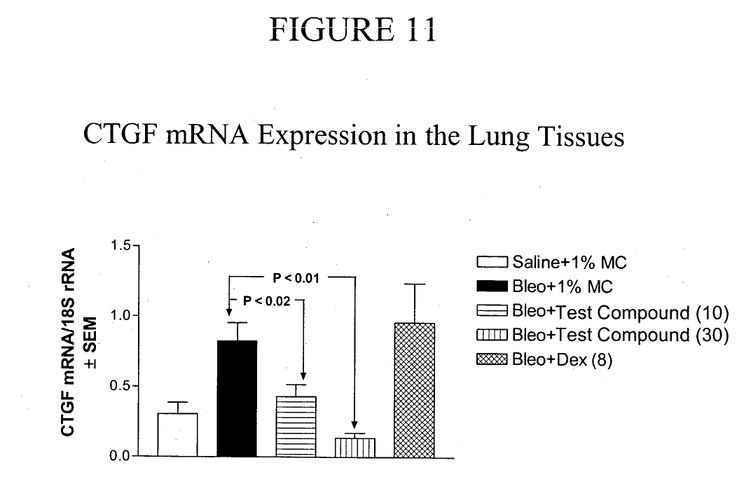

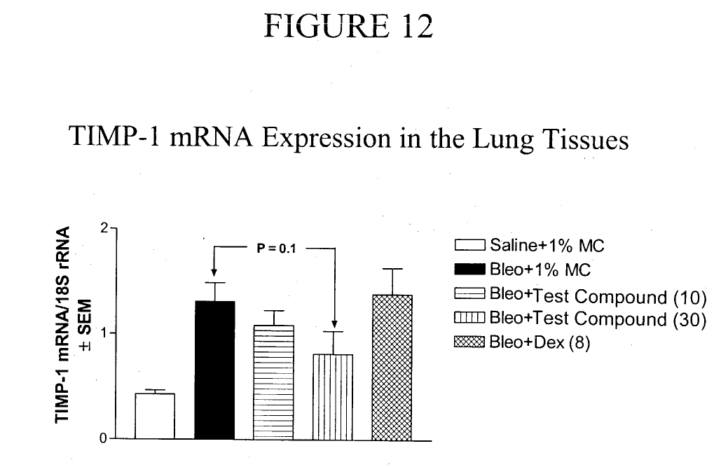

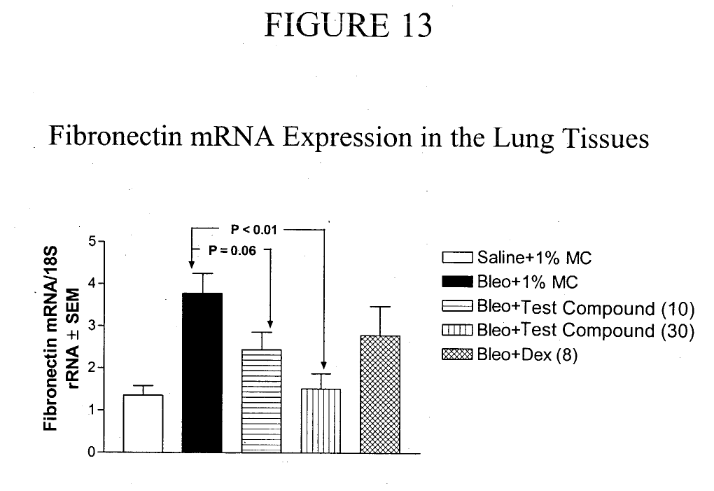

The results are shown in FIGS. 7-13. Bleomycin (Bleo) administration increased interleukin-6 (IL-6) levels in bronchoalveolar lavage fluids (BALF) (p<0.001) (FIG. 9). BALF IL-6 levels were significantly decreased by the treatment of 30 mg/kg of the TGF-β inhibitor test compound (p<0.01) or dexamethasone (Dex) (p<0.001) (FIG. 9). Furthermore, treatment of 30 mg/kg of the test compound, but not dexamethasone treatment, was associated with reduced levels of TGFβ-associated pulmonary mRNAs including PAI-1 (p<0.06) (FIG. 10), CTGF (p<0.01) (FIG. 11), TIMP-1 (p<0.1) (FIG. 12) and fibronectin (p<0.01) (FIG. 13).

In a similar experiment, synergistic effect of dexamethasone (Dex) and a representative test compound from compounds listed above in the bleomycin rat model of pulmonary fibrosis was studied. Rats were intubated with saline or 1 unit of bleomycin. After twenty four hours, rats were weighed and dosed with saline or 2.0 mg/kg of dexamethasone every other day and 1% methyl cellulose (MC) or 40 mg/kg of a test compound twice a day. On day 14, 1-3 hours after dosing, rat were sacrificed and lungs were inflated and collected for analysis.

These data support a synergistic effect between dexamethasone and the test compound. The primary end points of the analysis were the total hydroxyproline concentration per lung and lung capacity. The secondary end point was the body weight.

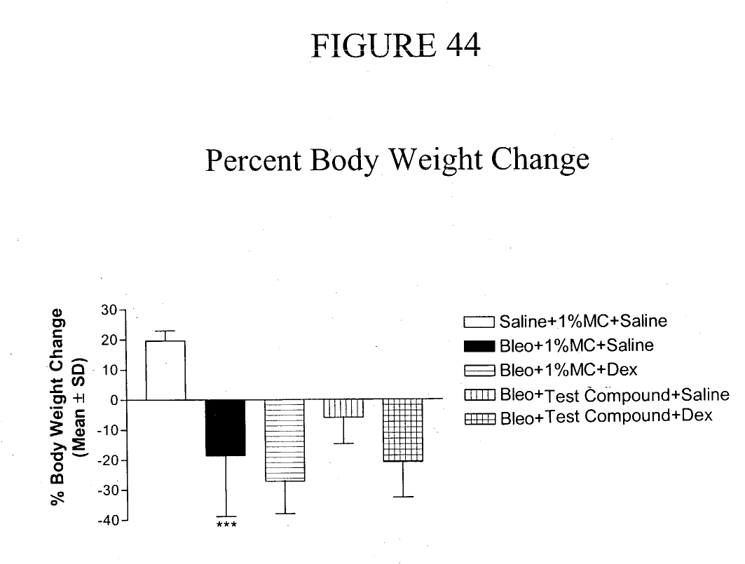

The results are shown in FIGS. 44-46. The statistical analysis was done using one-way ANOVA with Bonferroni's Multiple Comparison Test.

FIG. 44 shows that bleomycin administration induces a significant body weight loss (p<0.001), while treatment with Dex, the test compound, or Dex combined with the test compound has no effect on the bleomycin-induced body weight loss.

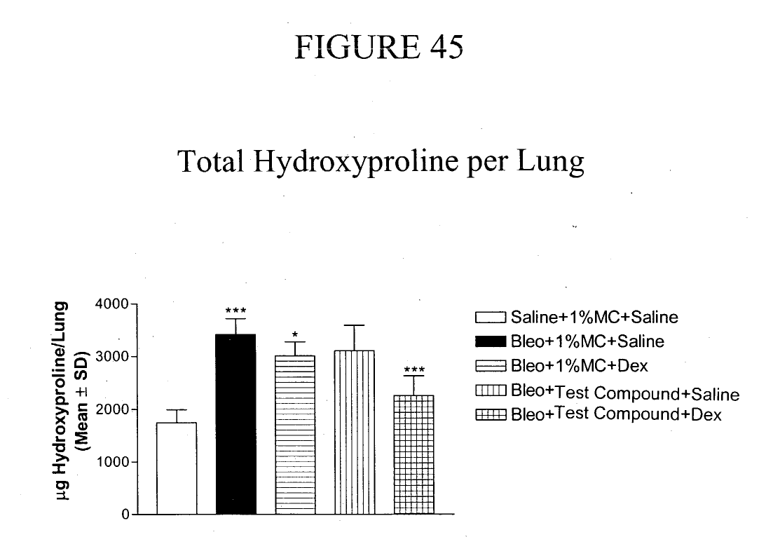

FIG. 45 shows that bleomycin administration induces a significant increase in total hydroxyproline in the lung (p<0.001). In addition, the Figure shows that the treatment with Dex (p<0.05), and Dex combined with the test compound (p<0.001) significantly decreases the total hydroxyproline concentration in the lung, induced by bleomycin. In particular, the treatment with Dex combined with the test compound shows significantly less total hydroxyproline in the lung than the treatment with Dex or the test compound alone (p<0.001).

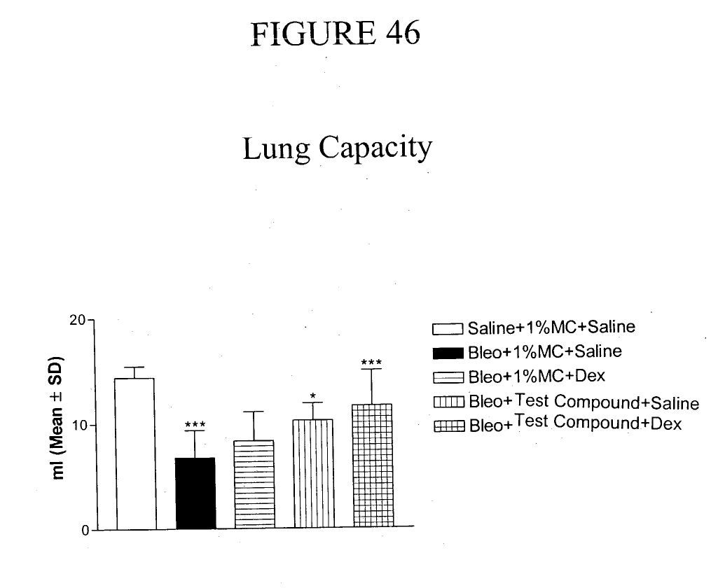

FIG. 46 shows that bleomycin administration induces a significant decrease in lung capacity (p<0.001). In addition, the Figure shows that the treatment with the test compound and Dex combined with the test compound significantly increases lung capacity which was reduced by bleomycin. In particular, the treatment with Dex combined with the test compound shows a significantly higher lung capacity than the lung capacity achieved by administration of Dex alone (p<0.05).

There is a trend in reducing bleomycin induced lung fibrosis with Dex (reduction in hydroxyproline, p<0.05), or with the test compound (increase in lung capacity, p<0.05) treatment alone. The combination treatment of Dex and the test compound significantly reduces bleomycin-induced lung fibrosis (reduction in hydroxyproline, and increase in lung capacity, p<0.001).

All references cited throughout the specification are expressly incorporated herein by reference. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, and the like. All such modifications are within the scope of the claims appended hereto.

What is claimed is:

1. A method for the treatment of a fibroproliferative disease, comprising
(a) identifying a non-peptide small molecule, selectively binding to a TGFβ-R1 kinase receptor; and
(b) administering an effective amount of said molecule to a mammalian subject diagnosed with said fibroproliferative disease.

2. The method of claim 1 wherein said fibroproliferative disease is a renal, hepatic, pulmonary, cardiovascular, eye, or dermatological disorder associated with enhanced TGF-β activity and excessive fibrosis or sclerosis.

3. The method of claim 1 wherein said fibroproliferative disease is selected from the group consisting of glomerulonephritis (GN); diabetic nephropathy; renal interstitial fibrosis; renal fibrosis resulting from complications of drug exposure; HIV-associated nephropathy; transplant necropathy; liver cirrhosis due to all etiologies; disorders of the biliary tree; hepatic dysfunction attributable to infections; pulmonary fibrosis; adult respiratory distress syndrome (ARDS); chronic obstructive pulmonary disease (COPD); idiopathic pulmonary fibrosis (IPF); acute lung injury (ALI); pulmonary fibrosis due to infectious or toxic agents; congestive heart failure; dilated cardiomyopathy; myocarditis; vascular stenosis; progressive systemic sclerosis; polymyositis; scleroderma; dermatomyositis; fascists; Raynaud's syndrome, rheumatoid arthritis; proliferative vitreoretinopathy; fibrosis associated with ocular surgery; and excessive or hypertrophic scar or keloid formation in the dermis occurring during wound healing resulting from trauma or surgical wounds.

4. The method of claim 1 wherein said molecule additionally inhibits a biological activity mediated by p38 kinase.

5. The method of claim 1 wherein said molecule preferentially inhibits a biological activity mediated by TGF-β -RI kinase relative to a biological activity mediated by p38 kinase.

6. The method of claim 1 wherein said molecule is a compound of formula (1) 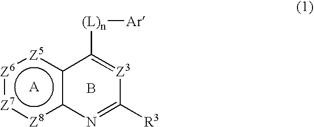
and the pharmaceutically acceptable salts and prodrug forms thereof
wherein R³ is a noninterfering substituent;
each Z is CR² or N, wherein no more than two Z positions in ring A are N, and wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents.

7. The method of claim 6 wherein said compound is a quinazoline derivative.

8. The method of claim 7 wherein Z³ is N; and Z⁵-Z⁸ are CR².

9. The method of claim 7 wherein Z³ is N; and at least one of Z⁵-Z⁸ is nitrogen.

10. The method of claim 7 wherein R³ is an optionally substituted phenyl moiety.

11. The method of claim 10 wherein R³ is selected from the group consisting of 2-, 4-, 5-, 2,4- and 2,5-substituted phenyl moieties.

12. The method of claim 11 wherein at least one substituent of said phenyl moiety is an alkyl(1-6C), or halo.

13. The method of claim 1 wherein said molecule is a compound of formula (2) 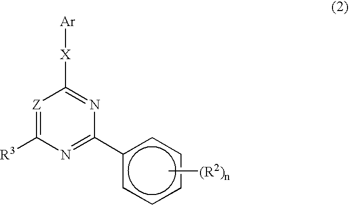
and the pharmaceutically acceptable salts and prodrug forms thereof; wherein
Ar represents an optionally substituted aromatic or optionally substituted heteroaromatic moiety containing 5-12 ring members wherein said heteroaromatic moiety contains one or more O, S, and/or N;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5.

14. A method for reversing the effect of TGF-β-mediated cell activation on the expression of a gene associated with fibrosis, comprising contacting a cell or tissue in which the expression of said gene is altered as a result of TGF-β-mediated cell activation, with a non-peptide small molecule inhibitor of TGF-β, specifically binding a TGFβ-R1 receptor kinase present in said cell or tissue.

15. The method of claim 14 wherein said gene is associated with fibrosis.

16. The method of claim 15 wherein said gene is overexpressed as a result of TGF-β-mediated cell activation.

17. The method of claim 16 wherein said gene is selected from the group consisting of fibronectin, collagen, type I, alpha 2 COL1A2); collagen, type V, alpha 2 (COL5A2); connective tissue growth factor (CTGF); thrombospondin 1 (THBS 1); hexabrachion (HXB); tissue inhibitor of metalloproteinase 1 (TIMP-1); tissue inhibitor of metalloproteinase 3 (TIMP3); plasminogen activator inhibitor-1 (PAI-1); and collagen, type III, alpha 1 (COL3A1).

18. The method of claim 17 wherein said inhibitor reverses the effect of TGF-β-mediated cell activation on the expression of two or more of said genes.

19. The method of claim 18 wherein said gene is underexpressed as a result of TGF-β-mediated cell activation.

20. The method of claim 19 wherein said gene is platelet-derived growth factor receptor-α (PDGFRα).

21. The method of claim 20 wherein said inhibitor reverses the effect of TGF-β-mediated cell activation on the expression of two or more of said genes.

22. The method of claim 21 wherein said tissue is selected from the group consisting of lung tissue, heart tissue, liver tissue, and kidney tissue.

23. The method of claim 22 wherein said inhibitor reverses the effect of TGF-β-mediated cell activation on a multiplicity of genes associated with fibrosis.

24. The method of claim 14 wherein said inhibitor additionally blocks biological activities mediated by Smad proteins, p38 and TAK1.

25. The method of claim 14 wherein said inhibitor is of the formula 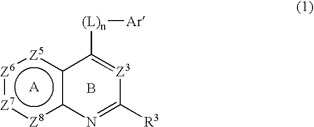
and the pharmaceutically acceptable salts and prodrug forms thereof
wherein R³ is a noninterfering substituent;
each Z is CR² or N, wherein no more than two Z positions in ring A are N, and wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents.

26. The method of claim 25 wherein said compound is a quinazoline derivative.

27. The method of claim 26 wherein Z³ is N; and Z⁵-Z⁸ are CR².

28. The method of claim 27 wherein Z³ is N; and at least one of Z⁵-Z⁸is nitrogen.

29. The method of claim 27 wherein R³ is an optionally substituted phenyl moiety.

30. The method of claim 29 wherein R³ is selected from the group consisting of 2-, 4-, 5-, 2,4- and 2,5-substituted phenyl moieties.

31. The method of claim 30 wherein at least one substituent of said phenyl moiety is an alkyl(1-6C), or halo.

32. The method of claim 14 wherein said molecule is a compound of formula (2) 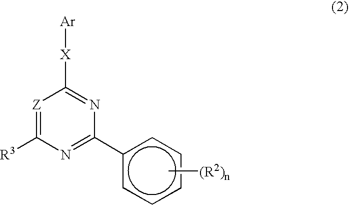
and the pharmaceutically acceptable salts and prodrug forms thereof; wherein
Ar represents an optionally substituted aromatic or optionally substituted heteroaromatic moiety containing 5-12 ring members wherein said heteroaromatic moiety contains one or more O, S, and/or N;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5.

33. A method for determining the likelihood of a positive response of a subject diagnosed with a fibroproliferative disease associated with TGF-β-mediated cell activation to treatment with a TGF-β inhibitor specifically binding the TGFβ-R1 receptor, comprising
(a) determining in a biological sample obtained from said subject the expression level of one or more genes selected from the group consisting of fibronectin (FN), collagen, type I, alpha 2 COL1A2); collagen, type V, alpha 2 (COL5A2); connective tissue growth factor (CTGF); thrombospondin 1 (THBS 1); hexabrachion (HXB); tissue inhibitor of metalloproteinase 1 (TIMP-1); tissue inhibitor of metalloproteinase 3 (TIMP3); plasminogen activator inhibitor-1 (PAI-1); platelet-derived growth factor receptor-α (PDGFRα); glucocorticoid receptor (GR); Smad2; Smad3; Smad4; Smad7; Col 1; Col 3; TGF-β activated kinase (TAKI); p38 alpha; β-actin; Cox1; Cox 2; I kappa-B kinase (iKKi); and collagen, type III, alpha-1 (COL3A1), compared with expression in a sample obtained from a normal subject; and
(b) indicating a positive response, if one or more of said genes are differentially expressed.

34. The method of claim 33 wherein a positive response is indicated if one or more genes selected from the group consisting of fibronectin (FN), collagen, type I, alpha 2 (COL1A2); collagen, type V, alpha 2 (COL5A2); connective tissue growth factor (CTGF); thrombospondin 1 (THBS 1); hexabrachion (HXB); tissue inhibitor of metalloproteinase 1 (TIMP-1); tissue inhibitor of metalloproteinase 3 (TIMP3); plasminogen activator inhibitor-1 (PAI-1); Smad7; Col 1; interleukin-6 (IL-6); Cox1; Cox2; and collagen, type III, alpha 1 (COL3A1) are overexpressed.

35. The method of claim 33 wherein a positive response is indicated if one or more of genes selected from the group consisting of platelet-derived growth factor receptor-α (PDGFRα); glucocorticoid receptor (GR); Smad3; and I kappa-B kinase (iKKi) are underexpressed.

36. The method of claim 33 wherein said fibroproliferative disease is selected from the group consisting of glomerulonephritis (GN); diabetic nephropathy; renal interstitial fibrosis; renal fibrosis resulting from complications of drug exposure; HIV-associated nephropathy; transplant necropathy; liver cirrhosis due to all etiologies;
disorders of the biliary tree; hepatic dysfunction attributable to infections; pulmonary fibrosis; adult respiratory distress syndrome (ARDS); chronic obstructive pulmonary disease (COPD); idiopathic pulmonary fibrosis (TPF); acute lung injury (ALI); congestive heart failure; dilated cardiomyopathy; myocarditis; vascular stenosis; progressive systemic sclerosis; polymyositis; scleroderma; dermatomyositis; fascists; Raynaud's syndrome, rheumatoid arthritis; proliferative vitreoretinopathy; fibrosis associated with ocular surgery; and excessive or hypertrophic scar or keloid formation in the dermis occurring during wound healing resulting from trauma or surgical wounds.

37. A method of diagnosing a patient with a fibroproliferative disease, comprising
(a) determining in a biological sample obtained from said patient the expression level of one or more genes selected from the group consisting of fibronectin, collagen, type I, alpha 2 (COL1A2); collagen, type V, alpha 2 (COL5A2); connective tissue growth factor (CTGF); thrombospondin 1 (THBS1); hexabrachion (HXB); tissue inhibitor of metalloproteinase 1 (TIMP-1); tissue inhibitor of metalloproteinase 3 (TIMP3); plasminogen activator inhibitor-1 (PAI-1); collagen, type III, alpha 1 (COL3A1); glucocorticoid receptor (GR); Smad2; Smad3; Smad4; Smad7; Col 1; Col 3; TGF-β activated kinase (TAKI); p38 alpha; β-actin; Cox1; Cox 2; I kappa-B kinase (iKKi); and platelet-derived growth factor receptor-α (PDGFRα), compared with expression in a normal sample; and
(b) diagnosing said patient with a fibroproliferative disease if one or more of said genes are differentially expressed.

38. The method of claim 37 wherein said patient is diagnosed with said fibroproliferative disease if one or more genes selected from the group consisting of fibronectin (FN), collagen, type I, alpha 2 (COL1A2); collagen, type V, alpha 2 (COL5A2); connective tissue growth factor (CTGF); thrombospondin 1 (THBS1); hexabrachion (HXB); tissue inhibitor of metalloproteinase 1 (TIMP-1); tissue inhibitor of metalloproteinase 3 (TIMP3); plasminogen activator inhibitor-1 (PAI-1); Smad7; Col 1; interleukin-6 (IL-6); Cox1; Cox2; and collagen, type III, alpha-1 are overexpressed.

39. The method of claim 37 wherein said patient is diagnosed with a fibroproliferative disease if one or more of genes selected from the group consisting of platelet-derived growth factor receptor-α (PDGFRα); glucocorticoid receptor (GR); Smad3; and I kappa-B kinase (iKKi) are underexpressed.

40. A method of treating a fibroproliferative disease in a subject, comprising administering to the subject dexamethasone and a TGF-β inhibitor, specifically binding a TGFβ-R1 receptor.

41. The method of claim 40 wherein the fibroproliferative disease is a renal, hepatic, pulmonary, cardiovascular, eye, or dermatological disorder associated with enhanced TGF-β activity and excessive fibrosis or sclerosis.

42. The method of claim 40 wherein said fibroproliferative disease is selected from the group consisting of glomerulonephritis (GN); diabetic nephropathy; renal interstitial fibrosis; renal fibrosis resulting from complications of drug exposure; HIV-associated nephropathy; transplant necropathy; liver cirrhosis due to all etiologies; disorders of the biliary tree; hepatic dysfunction attributable to infections; pulmonary fibrosis; adult respiratory distress syndrome (ARDS); chronic obstructive pulmonary disease (COPD); idiopathic pulmonary fibrosis (IPF); acute lung injury (ALI); pulmonary fibrosis due to infectious or toxic agents; congestive heart failure; dilated cardiomyopathy; myocarditis; vascular stenosis; progressive systemic sclerosis; polymyositis; scleroderma; dermatomyositis; fascists; Raynaud's syndrome, rheumatoid arthritis; proliferative vitreoretinopathy; fibrosis associated with ocular surgery; and excessive or hypertrophic scar or keloid formation in the dermis occurring during wound healing resulting from trauma or surgical wounds.

43. The method of claim 40 wherein the subject is human.

44. The method of claim 43 wherein said TGF-β inhibitor is a compound of formula (1) 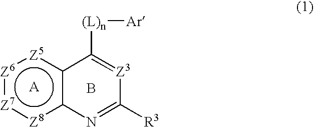
and the pharmaceutically acceptable salts and prodrug forms thereof
wherein R³ is a noninterfering substituent;
each Z is CR² or N, wherein no more than two Z positions in ring A are N, and wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents.

45. The method of claim 43 wherein said TGF-β inhibitor if a compound of formula (2) 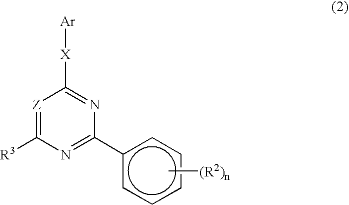
and the pharmaceutically acceptable salts and prodrug forms thereof; wherein
Ar represents an optionally substituted aromatic or optionally substituted heteroaromatic moiety containing 5-12 ring members wherein said heteroaromatic moiety contains one or more O, S, and/or N;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5.
